(19) United States
(12) Patent Application Publication
(10) Pub. No.: US 2005/0287230 A1
(43) Pub. Date: Dec. 29, 2005

(54) Method of producing ginsenoside 20 (R)-Rh2 and composition of matter thereof

(76) Inventor: Jeffrey Young, Alhambra, CA (US)

Correspondence Address: RAYMOND Y. CHAN, 108 N. YNEZ AVE., SUITE 128, MONTEREY PARK, CA, 91754, US

(21) Appl. No.: 10/965,942

(22) Filed: Oct. 14, 2004

Related U.S. Application Data
(60) Provisional application No. 60/582,765, filed on Jun. 25, 2004.

(51) Int. Cl. A61K 31/704; A61K 35/78

(52) U.S. Cl. 424/728; 514/26; 514/33

(57) ABSTRACT

The present invention is a method of producing ginsenoside comprising the steps of: (a) producing a predetermined quantity of ginseng dialcohol from an extraction process of ginseng dialcohol; (b) reacting the ginseng dialcohol with a predetermined quantity of acetobromo-α-D-glucose under a predetermined condition; and (c) obtaining a predetermined quantity of ginsenoside Rh₂. The present invention also provides a composition for enhancing the immune system of a living object comprising a quantity of ginsenoside, a quantity of ursolic acid and a quantity of shikonin. The composition may also comprise a quantity of panaxans, astragalan, and/or lentinan. The ginsenoside 20(R)-Rh₂, ursolic acid, shikonin, panaxans, astragalan and lentinan have an effective range between 0.001 mg/kg and 0.1 mg/kg, 0.5 mg/kg and 10 mg/kg, 0.5 mg/kg and 10 mg/kg, 0.1 mg/kg and 1 mg/kg, 0.1 mg/kg and 1 mg/kg, and 0.1 mg/kg and 1 mg/kg respectively.

### CROSS REFERENCE OF RELATED APPLICATION

This is a non-provisional application of a provisional application, application No. 60/582,765 filed on Jun. 25, 2004.

### BACKGROUND OF THE PRESENT INVENTION

1. Field of Invention

The present invention relates to a method of producing ginsenoside and a composition of matter thereof, and more particularly to a method of producing ginsenoside comprising a semi-synthesis process such that a high yield of ginsenoside is produced and that the ginsenoside is a major composition of the composition of matter of the present invention for cancer treatment.

2. Description of Related Arts

Ginseng is the root of panax ginseng belonging to the family Aralaceae Ginseng. The major active components of ginseng are the different varieties of ginsenosides which have the effects of promoting phagocytosis and enhancing lymphocyte-blastogenesis rate, combating cancer, decreasing the level of blood sugar, and promoting the health of human bodies, etcetera. The promissory effects of ginseng towards health lead to a high demand of ginseng and trigger thousands of researches. However, there is no existing satisfactory method of mass production of ginseng which is capable of meeting the need of the huge demand of ginseng. The price of ginseng flies higher and higher and it seems impossible to afford the payment for ginseng for the normal household in this country or in this world.

In the last several decades, a tremendous amount of effort has been made on anti-cancer drug research. However, the research for treating cancer is concentrated on the way to kill or remove the cancer cells. Thus, tens of thousands of these cells toxic drug researches have failed to escape the shadow of “killing the enemy, but also getting killed” that hangs over those annihilative drugs. When the cancer cells are killed or removed, the normal useful healthy cells are also killed or removed simultaneously. People become to realize the need for a different path or strategy other than that of the cell toxic drugs that kill or remove cancer cells. In recent years, far-reaching advances have been made in immunology, which has extended into biology and various fields of medical studies and renovated many conventional theories. The knowledge of immunology has been applied not only to geriatric health maintenance, ailment deterrence and health preservation in the field of preventive medicine, but also to combating certain major illnesses. With the discovery of immunological anti cancer drugs, a totally new and exciting frontier has been opened.

Statistics show that there are over two hundred million hepatitis B virus carriers in the world; in China and Southeast Asian alone, there are one hundred twenty million virus carriers. Each year, one million five hundred thousand people die from liver cancer. In the next twenty years, thirty million people will get liver cancer. Thirty thousand cases of hepatitis B are discovered in the US each year. The number of hepatitis C patients is three to four times higher than that of hepatitis B. This has caught the attention of the medical world.

The long history of clinical practice shows that the means of traditional Chinese medicine cannot bring about breakthroughs in the complete treatment of cancer. We must utilize all means evolved from the modern cell biology and develop natural medicine through molecular biology. Modern science and technology show that in the complex process of life there exist various adjusting organic mechanisms, such as the neurological endocrine and immunology systems, we have identified and separated micro matters, such as the neurohumor transmitters, enzymes, and receptors, that perform the adjustments. The neurohumor transmitter and hormones are the main carriers of neurological messages. It is critical for natural medicine to flow the path of seeking out living matter s that truly perform the adjusting functions by selecting natural herbs the immunological power of which is indicated by the receptors and the enzymes.

### SUMMARY OF THE PRESENT INVENTION

A main object of the present invention is a method of producing ginsenoside comprising a semi-synthetic process such that a high yield of ginsenoside is obtained.

Another object of the present invention is a method of producing ginsenoside comprising an extraction process of ginseng dialcohol and a semi-synthetic process of ginsenoside-Rh2 such that the cost of obtaining ginsenoside-Rh2 is greatly reduced for the consumer market.

Another object of the present invention is a composition of ginsenoside obtained by a method of producing ginsenoside which is capable of enhancing the immune system of human body.

Another object of the present invention is a composition for enhancing the immune system of a living object wherein said composition comprises a predetermined quantity of ginsenoside and a predetermined second composition selected from the group of ursolic acid, shikonin, panaxans, astragalan, and lentinan.

Another object of the present invention is a composition for enhancing the immune system of a living object wherein said composition comprises a predetermined quantity of ginsenoside synthesized by a method of producing ginsenoside such that the cost of obtaining said ginsenoside is greatly reduced and the time for producing said ginsenoside is shortened.

Another object of the present invention is a composition for enhancing the immune system of a living object wherein said composition comprises a predetermined quantity of ginsenoside synthesized by a method of producing ginsenoside and a second composition selected from the group of ursolic acid, shikonin, panaxans, astragalan, and lentinan.

Another object of the present invention is a composition for enhancing the immune system of a living object wherein said composition comprises a predetermined quantity of ginsenoside and a plurality of supplementary compositions selected from the group of ursolic acid, shikonin, panaxans, astragalan, and lentinan wherein said ginsenoside is produced by a method of producing ginsenoside comprising a semi-synthetic process such that the cost of producing the ginsenoside is greatly reduced and the time of producing the ginsenoside is shortened.

Another object of the present invention is a composition of enhancing the immune system of a living object wherein the composition comprises a predetermined quantity of ginsenoside which in turn introduces changes of a body condition of the living object such that a cell division or replication of a cancer cell is capable of being suppressed and that a degeneration of a cancer cell is capable of being induced.

Another object of the present invention is a composition of enhancing the immune system of a living object wherein the composition comprises a predetermined quantity of ginsenoside synthesized by a method of producing ginsenoside comprising an extraction process of ginseng dialcohol and a semi-synthetic process of ginsenoside-Rh₂ such that the cost of obtaining ginsenoside-Rh₂ is greatly reduced for the consumer market.

Another object of the present invention is a composition of enhancing the immune system of a living object wherein the composition comprises a predetermined quantity of ginsenoside synthesized by a method of producing ginsenoside comprising an extraction process of ginseng dialcohol and a semi-synthetic process of ginsenoside-Rh₂ such that the cost of obtaining ginsenoside-Rh₂ is greatly reduced for the consumer market, and that the composition is effective as being alternative for cancer treatment.

Another object of the present invention is a composition of enhancing the immune system of a living object comprising a predetermined quantity of ginsenoside and a predetermined plurality of supplementary compositions selected from the group of ursolic acid, shikonin, panaxans, astragalan, and lentinan, wherein the ginsenoside is synthesized by a method of producing ginsenoside comprising an extraction process of ginseng dialcohol and a semi-synthetic process of ginsenoside-Rh2 such that the cost of obtaining ginsenoside-Rh₂ is greatly reduced for the consumer market, and that the composition of the present invention is effective as being alternative for cancer treatment.

Another object of the present invention is a composition of enhancing the immune system of a living object comprising a predetermined quantity of ginsenoside and a predetermined plurality of supplementary compositions selected from the group of ursolic acid, shikonin, panaxans, astragalan, and lentinan, wherein the ginsenoside is synthesized by a method of producing ginsenoside comprising an extraction process of ginseng dialcohol and a semi-synthetic process of ginsenoside-Rh₂ such that the cost of obtaining ginsenoside-Rh₂ is greatly reduced for the consumer market, and that the composition of the present invention is effective as being alternative for treatment of liver cancer.

Accordingly, in order to achieve the above objects, the present invention is a method of producing ginsenoside comprising the steps of:
(a) producing a predetermined quantity of ginseng dialcohol from an extraction process of ginseng dialcohol;
(b) reacting the ginseng dialcohol with a predetermined quantity of acetobromo-α-D-glucose under a predetermined condition; and
(c) obtaining a predetermined quantity of ginsenoside Rh₂.

The extraction process of ginseng dialcohol comprises the steps of:
(a) extracting a predetermined quantity of n-butylalcohol from a red ginseng in a crude powder form with methanol;
(b) obtaining a predetermined quantity of total crude saponin from the n-butylalcohol under reduced pressure and steam dry conditions;
(c) refining the total crude saponin by adding 5% ethanol with 7% hydrochloric acid, heating under reflux hydrolysis for 4 hours and cooling under room temperature such that a hydrolyzed solution is obtained;
(d) extracting a total refined saponin from the hydrolyzed solution by diluting the hydrolyzed solution with water to form a diluted solution having 1.5 times volume of the hydrolyzed solution, removing the ethanol, and extracting by CHCl₃; and
(e) separating a predetermined quantity of ginseng trialcohol, a predetermined quantity of ginseng dialcohol, and a predetermined quantity of oleanolic acid from the total refined saponin by gradient cleansing using silica-gel column chromatography CHCl₂—MeOH (8:2).

The present invention also provides a composition for enhancing the immune system of a living object comprising a predetermined quantity of ginsenoside, a predetermined quantity of ursolic acid and a predetermined quantity of shikonin, wherein the ginsenoside is produced from the method of producing ginsenoside as described above. The composition may further comprise a predetermined quantity of panaxans, astragalan, and/or lentinan. The ginsenoside 20(R)-Rh₂, ursolic acid, shikonin, panaxans, astragalan and lentinan have an effective range between 0.001 mg/kg and 0.1 mg/kg, 0.5 mg/kg and 10 mg/kg, 0.5 mg/kg and 10 mg/kg, 0.1 mg/kg and 1 mg/kg, 0.1 mg/kg and 1 mg/kg, and 0.1 mg/kg and 1 mg/kg respectively.

These and other objectives, features, and advantages of the present invention will become apparent from the following detailed description, the accompanying drawings, and the appended claims.

### BRIEF DESCRIPTION OF THE DRAWINGS

FIG. 1A illustrates the chemical structure of ginsenoside 20(R)-Rh₂ of the present invention.

FIG. 1B illustrates the chemical structure of ursolic acid of the present invention.

FIG. 1C illustrates the chemical structure of shikonin of the present invention.

FIG. 1D illustrates the chemical structure of the three dextrans of panaxans A of the present invention.

FIG. 1E illustrates the chemical structure of the two dextrans of astragalan of the present invention.

FIG. 1F illustrates the chemical structure of lentinan of the present invention.

FIG. 2 is a flow diagram illustrating a semi-synthetic process of ginsenoside 20(R)-Rh₂ from panaxadiol of the present invention.

FIG. 3 is a flow diagram illustrating a reaction process of 20S-Protoganaxadiol to panaxadiol of the present invention.

FIG. 4 is a flow diagram illustrating an extracting and separating process of red ginseng of the present invention.

FIG. 5 is a flow diagram illustrating an extracting and separating process of ursolic acid of the present invention.

FIG. 6 is a flow diagram illustrating an extracting and separating process of shikonin of the present invention.

FIG. 7 is a flow diagram illustrating an extracting process of astragalan of the present invention.

FIG. 8 is a flow diagram illustrating an extracting and separating lentinan polysaccharide of the present invention.

FIG. 9 is a histogram illustrating the effect of a composition of experiment 5 of the present invention.

FIG. 10 is a histogram illustrating the results of the inhibitory effect of composition A of experiment 5 of the present invention.

FIG. 11 illustrates the effect of the composition A and interference substance on the activity of natural killer cells.

FIG. 12 illustrates the effect of the composition A and the interference substance on the activity of macrophage.

### DETAILED DESCRIPTION OF THE PREFERRED EMBODIMENT

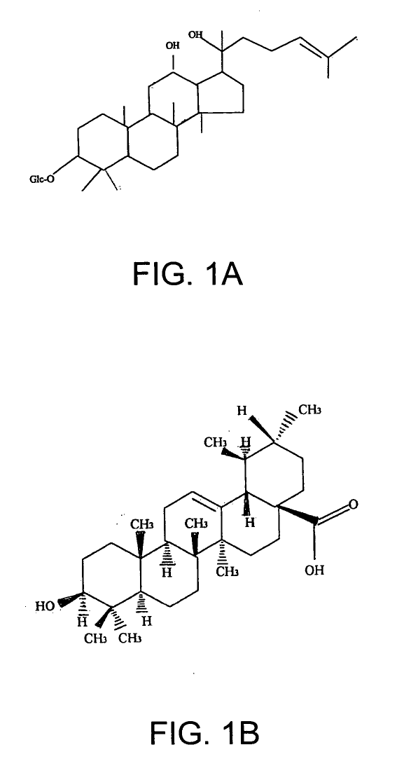

Referring to FIGS. 1A to 1F of the drawings, the chemical structures of ginsenoside 20(R)-Rh₂, ursolic acid, lentinan, panaxans A, astragalan, and lentinan are illustrated respectively.

The structure of ginsenoside 20(R)-Rh₂ is identified by a plurality of physical and chemical properties. The ginsenoside 20(R)-Rh₂ is a colorless needle-shaped crystal having a positive result in Liebermann-Burchard test. Hydrolysis testing results of thin layer chromatography shows that the aglycon is ginseng dihydrolic alcohol and the glycoside is glucose. In ¹H-NMR spectrum, the coincidental constant at the double peak δ4.94 ppm (1H) is 7 Hz, thus the glucose is β configuration. In ¹³C-NMR spectrum, C1′ signal is detected at δ106.9 ppm, and pentose signal, which is identical to methyl-β-D-glucopyranoside, is detected at δ78.8˜63.1 ppm. Further comparison of this chemical compound with the ¹³C-NMR spectrum of ginsenoside 20(S)-Rh₂ shows that the C17 and C21 signals of this chemical shift to a higher position by 4.2 ppm, while the C22 signal of this chemical shifts to a lower position by 7.9 ppm, thus showing that the C20 of this chemical is R configuration. Hence, this chemical is identified as ginsenoside 20(R)-Rh₂, and that the physical, chemical and optical founding are summarized as: (1) melting point (mp): 288˜290° C., optical rotation: [α]²⁷_{D}+21.1° (0.61/Methanol); (2) Theoretical value of element analysis: C: 67.45; H, 10.07, and actual value: C: 67.38; H: 10.16; (3) molecular formula: C₃₆H₆₂O₈.H₂O; (4) infrared spectrum [IR(KBr)cm⁻¹]: 3400(OH), 2970, 2940(CH₃, CH₂), 1630 (C═C), 1465, 1450 (CH₂), 1380 (CH₃), 1075, and 1020 (CO).

The structure of ginsenoside-Rh₂ is identified as ginseng dialcohol-3-O-β-D-glucopyranoside, which is a white powder, having melting point 264˜266° C., [α]²⁵_{D} positive (0.61/methanol), Liebermann-Burchard reaction positive (+), Moish reaction positive (+), and [IR(KBr)cm⁻¹]: 3400, 2790, 1450, 1385, and 1365.

The active components of ginseng have a variety of anti-tumor effects and properties, such as a combination of ginsenoside and cis-platinum aminochloride for posing inhibitive effect on tumor cells of oophoroma, a ginsenoside for posing inhibitive effect on tumor cells of Morris liver cancer, cervical cancer, and a variety of cancer diseases, a ginsenoside-Rh₂ for reversing the phenotype of culture cancer cells, a ginsenoside Rh₁ posing positive effect on tumor of Morris liver cancer or the like, a ginsenoside and a polysaccharides of ginseng for inhibiting growth of cancerous cells, a ginsenoside for anti-tumor having nontoxic nature. The above effects are further explained in the following description respectively.

A Combination of Ginsenoside and cis-Platinum Aminochloride for Posing Inhibitive Effect on Tumor Cells of Oophoroma

Ginsenoside Rh₂ (hereafter also named as Rh₂) externally inhibits the proliferation of oophoroma cells and the internal effectiveness of combining with cis-platinum aminochloride. Using the cell line in the ascites of the patient with critical ovary cystadenoma to study the inhibition effect of ginsenoside Rh₂ upon the growth of human oophoroma, ginsenoside Rh₂ external experiment inhibits the proliferation of HRA cells. Within the effective range of Rh₂ (10˜100 μmol/L), the extent of its inhibition has dosage-dependent connotation. When the dosage is applied more than 15 μmol, the inhibition extent of the synthesis of DNA, RNA and protein also has dependent relation. And then, with the grafting of Rh₂ inhibition into the mouse's HRA cells, its growth is not obvious. However, when the drugging is jointly conducted on both the cis-platinum aminochloride and the internal Rh₂ of 10 μmol/L, compare to the Non-Treatment Group, the Singly Given cis-platinum aminochloride Group or the Singly Given Rh₂ Group, growth of the tumor in the Combined Drugged Group is clearly inhibited. In fact, survival time is also significantly extended.

Other experiments also show, when the drugging is jointly used on ginsenoside Rh₂ and cis-platinum aminochloride, coordination effect emerges. During the experiment, the weight, blood cell volume and Rh₂ density are monitored but no side effect is found. It also reveals ginsenoside-Rh₂ has significant effect on inhibiting the tumor strain of promyelcytic leukemia HL60.

A Ginsenoside for Posing Inhibitive Effect on Tumor Cells of Morris Liver Cancer, Cervical Cancer, and a Variety of Cancer Diseases

Lab experiment shows, in the external experiment, ginsenoside Rh₂ which is the special composition of red ginseng, has outstanding inhibition on the proliferation function of Morris liver cancer, human cervical cancer, B16 malignant melanoma and Hela cells. In fact, ginsenoside Rh₂ and ginsenoside 20(R)-Rh₁ is able to inhibit the human promyelcytic leukemia HL60 tumor strain significantly. Ginseng ropargylalcohol has a certain inhibition effect on those externally cultured cells such as MK-1 cell, B16 cell, L929 cell, SW969 cell, Hela cell and K562 cell and their ED₅₀ are 0.8, 1.7, 2.2, 2.3, 10.7, 11 and 7 μg/ml respectively.

A Ginsenoside-Rh₂ for Reversing the Phenotype of Culture Cancer Cells

Study shows ginsenoside Rh₂ is able to deteriorate the phenotypic expression of cultured cancer cells. Its mechanism is: Rh₂ has strong affinity towards the lipid-bimolecular layer. It is able to enter the bimolecular layer of cytolipin. By changing the nature of the lipid-bimolecular layer, it affects the molecules that lead to various functions on the surface cancerous cells. For instance, it affects the glycosidase, the glucosyltransferase, the receptor protein and the adherent protein. Furthermore, Rh₂ changes the signals caused by these molecules or alters the transfer of these signals to the cell nucleus. As a result, it changes the expression of some of the genes, like the cancerous C-myc gene. Thus, cancer cells which have been dealt with by Rh₂, can succeed in obtaining the expression of the phenotype being close to normal cells.

Ginsenoside Rh₂ in the red ginseng (untreated fresh ginseng does not have this substance) has stronger anti-tumor activity. It also enables the cancerous cells to redifferentiate and induces them to reverse and become non-cancerous cells. In culturing the melanoma (B16) and the human red cells, after ginsenoside-Rh₂ has permeated the cytomembrane, determine the transformation of cytomembrane's polarization degree and the fluidity of cytomembrane is found to have improved. This effect may have to do with the deterioration of the cancerous cells. Red ginseng has the function of enabling the cancerous cells to redifferentiate and inducing them to deteriorate and become non-cancerous cells.

Study of ginseng enabling the cancerous cells to deteriorate is carried out. Using the culture medium containing 20 mg/ml or 100 mg/ml ginsenoside, culture and graft the rat's Morris liver cancerous cells. After 25 times of generational cultures, Morris liver cancerous cells are found to be undergoing morphological change. They are distinguishingly different from Morris liver cancerous cells. Microscopic examination finds them to be compact cells with monolayer which have very distinctive intercellular space which are typical epithelium cellular form. They are also similar to normal cells. This shows the cancerous cells of the liver have “deteriorated”. After 300 times of generational cultures, all the Morris liver cancerous cells have “deteriorated”.

Microscopic examination cannot find Morris liver cancerous cells anymore.

A Ginsenoside Rh₁ Posing Positive Effect on Tumor of Morris Liver Cancer or the Like

Laboratory study shows, although ginsenoside has no inhibition effect on the proliferation of Morris liver cancerous cells, it is able to activate the adenosine cyclic enzyme of Morris liver cancerous cells. The study further shows ginsenoside-Rh₁ has no inhibition effect on the proliferation of B16 melanoma cells but enables to promote the synthesis of the melanin of the cells. This synthesis is direct proportion to the dosage of ginsenoside Rh₁. It promotes the synthesis of melanin, even though when it is under a relatively lower cellular density. Ginsenoside enables the inverted transformation to happen in the culture cells of Morris hepatoma and has the function of leading them to normalize.

A Ginsenoside and a Polysaccharides of Ginseng for Inhibiting Growth of Cancerous Cells

Ginsenoside and ginseng polysaccharides have the effect of inhibiting the growth of cancerous cells. Experiments indicate ginseng has certain inhibition effect on liver cancer of rat, melanoma of mouse, sarcoma 180 of mouse and E's ascites cancer of mouse (EAC). Adding ginsenoside Rh₂ (2-10 μg/ml) to the culture solution that cultures the Morris liver cancerous cells, the melanoma cells of mouse and the Hela cells, ginsenoside Rh₂ is found to have different degrees of inhibition effect on the proliferation of these cancerous cells.

A Ginsenoside for Anti-Tumor Having Nontoxic Nature

Lab study shows the preparation containing ginsenoside, oleanolic acid, methylphenyl ether, harringtonine and high-harringtonine, has the curative effect on leukemia but no toxicity to the heart. It is not harmful to the hematopoietic organ. By using the preparation, which contains ginsenoside, methylphenyl ether, ferulic acid and cinnamic sodium, it can raise the cardiac muscle cells' survival rate processed with adriamycin. And, the harringtonine, the high-harringtonine and the adriamycin are anti-cancerous medication that can inhibit the marrow but are poisonous to the heart. Thus, the preparation, which contains ginsenoside has the effect of mitigating the toxicity of anti-cancerous medication.

The anti tumor nature of ginseng mainly includes two mechanisms. The first mechanism employs the effect of ginseng on the cancerous cells containing enzyme, the differentiation of the cancerous cells and proliferation cycle, while the second mechanism is the activation of immune defense system.

The first mechanism is further described as follows:

Experiment shows various monomers of ginsenoside, using the activity function of various enzymes which inhibits the proliferation of cancerous cells and the adenosine cyclic enzyme as index to compare the activity of inducing the redifferentiation of cancerous cells. The concept of redifferentiation of the cancerous cells includes two aspects: inhibiting the proliferation of cancerous cells and transforming the mechanism of cancerous cells. Ginsenoside Rh₂ has the inhibition effect upon the proliferation of each and every cancerous cells. However, although ginsenoside has no inhibitory effect on cancerous cells, such as the Morris liver cancerous cells, it is able to activate the adenosine cyclic enzyme of Morris liver cancerous cells. Consequently, it takes the major role in inducing the redifferentiation of cancerous cells. When ginsenoside-Rh₁ is above 8.0 μg/ml, it is able to completely inhibit the proliferation of B16 melanoma cells. The analysis of cytofluorescope and autoradiograph proves that inhibiting the proliferation of melanoma cells is not by way of inhibiting the cellular DNA synthesis, but by blocking the cellular proliferation during the proliferation cycle between the period of G₁ and S. Ginsenoside-Rh₁ can promote the melanin synthesis of the cells although it is unable to inhibit the proliferation of the cells of B₁₆ melanoma and induce its morphological transformation. Ginsenoside Rh₂ can only promote the synthesis of melanin when the cellular density is high, whereas, ginsenoside Rh₁ is still able to promote the synthesis of melanin even when it is under the condition of relatively low cellular density. Results of the repeated experiments of colony formation and cellular proliferation show the inhibition of ginsenoside-Rh₂ is not upon the cytoxin and cannot kill the cancerous cells directly. It is inferred that the integration of proliferation molecules and receptor of the cancerous cells is hindered.

The second mechanism is further explained as follows. The immune function of ginseng on the tumor is the chief mechanism function of ginseng's fighting tumor.

Until now, more than 30 kinds of ginseng polysaccharide have been obtained from the separation of ginseng. The experiment indicates these polysaccharides are able to enhance the phagocytic function of the reticuloendothelial system, stimulate the growth of the complement and the antibody in the blood, improve the content of immunoglobulin in the blood, inhibit the growth of experimental tumors such as S₁₈₀, EAC and U₁₄ and strengthen the anti-tumor effect of cyclophosphamide.

Lab study shows ginseng polysaccharide, not only able to strengthen the anti-tumor effect of cyclophosphamide, is also able to inhibit and resist the immune toxicity caused by the cyclophosphamide. Experiment also proves the ginseng polysaccharide can strengthen the phagocytic function of the normal animal's reticuloendothelial system.

It has the stimulant effect on the serum complement, IgG and the growth of antibody.

Ginseng polysaccharide has similar stimulant effect on the phagocytic function of the cancerous mouse's reticuloendothelial system. Ginseng polysaccharide has no direct killing effect on the tumor cells and no obvious effect on the synthesis of DNA and RNA of tumor histology either.

Ginsenoside is able to heighten the phagocytic function of the mouse's reticuloendothelial system. Six days after the intra-abdominal injection, the phagocytic function of the mouse's subcutaneous connective tissue is very active. The examination of histological chemistry discovers the activity of mitochondria is intensified, which means the metabolism is exuberant. Increasing the dosage does not bring about the effectiveness instead. The immune function of the cancerous mouse decreases. Orally taking ginsenoside (50 mg/kg) enables its phagocytic function, formation of hemolysin and delayed ultrasensitive reaction to recover with certainty. Total saponin of ginseng is able to raise the cAMP and cGMP content of the lymphatic cells and accelerate the E-rosette formation. It is yet able to raise the complement content in the guinea pig's serum and the immunoglobulin content in the mouse's serum.

Ginsenoside has a certain inhibition effect upon the mouse's sarcoma S₁₈₀ and E's ascites cancer (EAC). It can enhance the anti-tumor function of cyclophosphamide too. Ginsenoside has significant effect on the regulatory net of the natural cell-killing interferon, the white cell medium-2. It is capable of reducing the cancerous rate of the mouse, enhancing its NK activity (P>0.02) and raising the production of IFNr and Il-2 to the level higher than the Contrast Group. According to the report of scholar, ginsenoside-Ra1 enables the activity of tumor necrosin (rhTNF-a) of man to recombine and the external experiment Ra1 to increase the rhTNF-a activity to 134 times more. The internal experiment also proves that it is able to intensify the anti-tumor activity of rhTNF-a.

Study of the effect of ginsenoside Re and the total saponin of American ginseng upon the immune function of the tumor patient shows ginseng saponin Re and the total saponin of American ginseng is clearly able to enhance, within the dosage range of fixed density, the activity of NK and LAK cells of the tumor patient. Enhancement effect of gingsenoside Re upon the NK activity of the tumor patient increases according to the dosage and the activity of NK cells obviously increases. Effect of ginsenoside Re upon the LAK activity of tumor patient only appears when it is above the 5 μg/ml level. It does not function when it is within the range of low dosage (p>0.05).

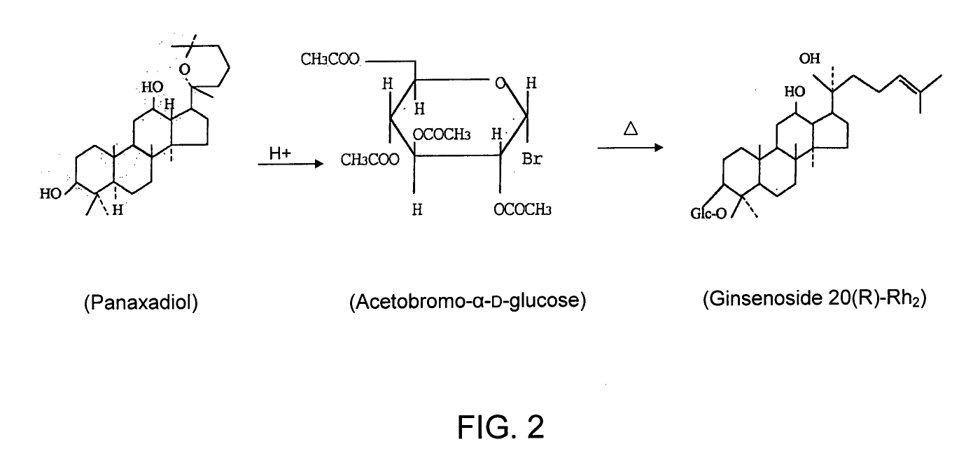

Referring to FIG. 4 of the drawings, an extracting and separating process of red ginsenoside of the present invention is illustrated. Red ginseng crude powder is used, which is then undergo methanol extraction to form a methanol liquid extract. Then the methanol is collected and water is added with a saturated water n-butylalcohol extraction and separation to form a water liquid and a n-butylalcohol liquid. The n-butylalcohol liquid is then steam dry under reduced pressure and the crude product of total saponins is obtained. After adding 5% ethanol containing 7% hydrochloric acid and heating under reflux condition for four hours for hydrolytic reaction, cool down to obtain a hydrolyzed liquid. The hydrolyzed liquid is then added with water for having a volume which is 1.5 times of the original volume. Remove ethanol and undergo CHCl₃ extraction for a resulting total saponins. Silica gel chromatography with CHCl₃-MeOH gradient elution is then used for separation of the total saponins. Ginseng trialcohol (panaxatriol), ginseng dialcohol (panaxadiol), and oleanolic acid are separated and resulted.

Referring to FIG. 1B of the drawings, the chemical structure of ursolic acid is illustrated. The physical and chemical properties of ursolic acid are further described. Ursolic acid is a white needle-shaped crystal in ethanol, having a melting point 277˜279° C. (291° C., 285˜288° C.), [α]³¹_{D} is +65.3° (0.45/Methanol), highly soluble in hexacyclic dioxide and pyridine, soluble in methanol, ethanol, butanol, and butanone, slightly soluble in propanone, very slightly soluble in benzene, chloroform, and ethyl ether, and insoluble in water and petroleum ether.

Ursolic acid has anti cancerous effect, and has a very outstanding inhibition rate in culturing the external liver cells. It is able to extend the life of mouse with Ehrlich's ascites cancer.

Ursolic acid also exerts protective effect towards liver damage. Experiment indicates subcutaneous injection of Ursolic acid (10 mg/kg) can reduce the serum gorge invert ammonia enzyme of the mouse's acute liver damage caused by carbon tetrachloride. Compare to Contrast Group (which is equivalent to control group), the difference is obvious. Determination result of primary content of hepatic sugar shows primary content of hepatic sugar of animal with acute liver damage lowers significantly. Having been treated with Ursolic acid, hepatic sugar of the animal clearly increases. Meanwhile, it also proves, after the medical treatment, content of animal's liver triglyceride reduces significantly and content of serum triglyceride and β-fattiprotein increases. The above result points out, Ursolic acid inclines to have fat-drawn effect. Research of liver histology indicates, after the animal has been treated with Ursolic acid, comparing to the Contrast Group, denaturation and necrosis of the liver cells mitigate significantly. Most of it returns to normal, except some of the animal's liver still has light denaturation.

The toxicity effect of ursolic acid is illustrated by having a LD₅₀ 680 mg/kg of mouse having abdominal injection.

The testing of ursolic acid is described further. Weigh accurately the powder sample of leaf of Ligustrum lucidum (20 g) and place it in the Soxhlet extractor. Add 300 ml of ethanol, extract, during the circular flow, until it becomes colorless. Retrieve the solvent until it is dry. Leftover, added with petroleum ethyl, soaks twice. 15 ml each time. Soaked for about 2 minutes. Exhaust the petroleum ethyl. Warm the mixed liquid of water-free ethanol ethoxy ethane (2:3) until it dissolves, settles at 5 ml capacity flask and kept in reserve. On thin layer of the same silicon sheet G-CMC, absorb accurately the standardized liquid of specimen liquid at intersection. Spread out with Cyclohexane-Chloroform-Ethyl Acetate (20:5:8). Solvent of 5% sulphuric ethanol is heated for 15 minutes at 110° C. until color appears.

Zigzag scan of twin-wavelength reflexibility, Sx=3.0, loophole 1.20 mm×1.20 mm, λs=520 nm, λ_{R}=700 nm. Within the scope of 1˜6 μg, Ursolic acid shows linear relation.

The anti tumor effect of ursolic acid is illustrated. Through the external model and chemical luminescent method, study the inhibition effect of the mouse's adrenal tumor cells (Rerca) and the mouse's bladder tumor cells (MBT) upon the function of system J₇₄₄ of the mouse's macrophage (MΦ). Explore the deterioration effect of Ursolic acid upon this inhibition effect. Ursolic acid (50 μg and 100 μg) is able to partially deteriorate Rerca cells to induce the inhibition effect of MΦ function. Ursolic acid 50 μg or 100 μg can bring about the oxidation luminescence (CL) of the J₇₄₄ cells caused by the upper liquid's free cells to return to normal level. Ursolic acid 100 μg can totally or partially restore the MΦ inhibition induced by the MBT-2 cells. It can also restore the J₇₄₄CL to a level higher than the normal. 50 μg/ml Ursolic acid partially deteriorates the inhibition function of MΦ, whereas, 100 μg/ml Ursolic acid a can totally deteriorate the inhibition function of MΦ. The outcome shows: Ursolic acid, through tumor cell's deteriorating the inhibition function of MΦ, develops the effect of resisting the tumor. Ursolic acid has an important role in adjusting the host defense mechanism. In the chemical luminescence manifestation of the mouse's macrophage system J₇₄₄, Ursolic acid acts as indicator effect of the macrophage function. Experiment of Ursolic acid shows obvious enhancement effect of chemical luminescence-related dosage. When it is combined with western medicines, coordinated enhancement effect is produced and growth of the mouse's experimental transitional cell carcinoma is significantly inhibited. OLA is able to stimulate T cells to secrete IL-2 and strengthen, to a certain extent, the proliferation effect of mitoelement PHA, cona, PWM and IL-2 upon the lymphatic cells of ordinary people and patient with malignant tumor.

The toxicity of ursolic acid is described further. Toxicity of the product on animal is very minor. One shot of fresh and ripe fruit, weighing 75 grams given to the rabbit, shows no toxic reaction. No toxic side effect is found after treatment dosage is applied. Large rat is drugged consecutively for 50 days. 17-ketone steroid level in the urine and Contrast Group's show no difference. Secondary acute toxicity experiment, intra-abdominal injection of rabbit is given with Ursolic acid's total abstract 50 mg/kg. The drugging continues for 6˜12 weeks. No abnormality is found in the heart, liver or kidney. OLA examines and finds LD₅₀ of the mouse's one-time subcutaneous drugging is 340 mg/kg. No toxicosis death is found in the mouse which has received 1 g/kg and 5 mg/20 g orally and by way of subcutaneous injection respectively. With acute toxicity tests, Ursolic acid's total abstract of larger rat's intravenous injection is 50 mg/kg and the smaller mouse's 5 mg/29 g. After 24 hours of observation, no harmful effect is found. Large rat is orally given with OLA 180 mg/kg once a day and continues for 10 days. On the 11th day, head is severed for material selection. Brain, heart, lungs, liver, spleen, thyroid gland, testicle, stomach, small intestine (4˜5 cm below the stomach) and bladder are examined under the microscope and no apparent damages are found in these organs.

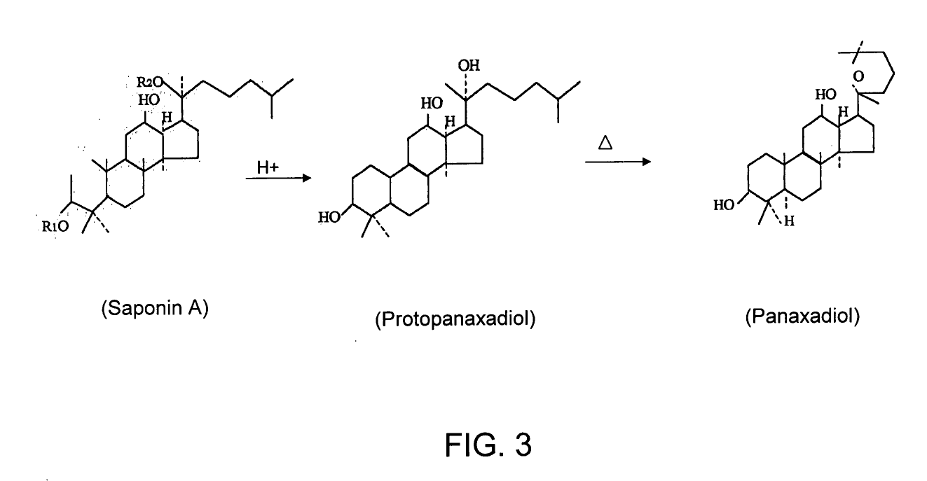

Referring to FIG. 5 of the drawings, an extracting and separating process of ursolic acid of the present invention is illustrated. Dry powder of leaf of Ligustrum lucidum, in the amount of 1 kg, is used. Ethanol, in the quantity of 6 times of the dry powder, is used for reflux extraction for 5 times, each lasting for 2 hours. Filter while the solution is hot to obtain a filtrate liquid. After concentrating the liquid to 800 ml with reduced pressure, place the liquid for a night and filter the liquid to obtain a filtrate and a sediment containing a ursolic acid ointment. The sediment is then washed with 300 ml petroleum ethyl at 60° C. to 80° C. and a solid matter is obtained. The solid matter is then dissolved in ethanol, decolorized with active carbon, and undergoes filtration while hot to obtain a filtrate liquid. The filtrate liquid is then cool to cool colloid state, which is then extracted and filtered into a white solid and a filtrate liquid. Placing the filtrate liquid and a pure ursolic acid which is a colorless needle form crystal is formed.

Referring to FIG. 1C of the drawings, the chemical structure of shikonin is illustrated. Shikonin is in the forms of purple crystal strip or crystal powder, having melting point 147˜149° C., [α]²⁰_{D}+138° (benzene), insoluble in water, soluble in alcohol, organic solvent, and vegetable oil.

The anti tumor effect of shikonin is examined. The mouse's abdomen is injected with Shikonin 10 mg/(kg·d). It has absolute inhibition effect upon the growth of S180 ascites cells. Lengthening rate of the animal's life is 92.5%.

According to a report, Shikonin is able to increase the radioactive treatment effect on the mouse's liver cancer and Lewis lung cancer. Based on the observation of Shikonin's radioactively magnified sensitiveness upon the mouse's liver cancer and the Lewis lung cancer, it is discovered that the anti-tumor rate increases, growth of the tumor is delayed and survival time of the mouse prolongs relatively. Under microscopic observation, the necrosis mess in the cancerous tissues of Lewis lung cancer is relatively larger. Ultrastructure manifests obvious existence of macrophage among the cancerous cells. It shows Shikonin has certain magnified effect of radiation upon liver cancer and Lewis lung cancer. The combination of Shikonin and radiation is able to intensify the mouse's macrophage of Lewis lung cancer's immediate killing power upon the cancerous cells.

The internal metabolism of shikonin is also examined. After the intravenous injection of H-Shikonin, the blood plasma's half-life phase of elimination (T_{1/2} β) is 8.79 h and the volume of distribution 8.91 L/kg. After the intravenous injection, content in the bile and liver is the highest, in the lungs, spleen, kidney, heart and skin the medium, in the testicle, muscle and brain the lowest. After the muscle injection and stomach irrigation, the absorption is rapid. The peak reaching time of blood plasma density is 7.62 min and 5.78 min. After the intravenous injection, only a small portion discharges in medication true form, in urine and in stool. Most of it discharges in changed form.

The shikonin is also examined by thin layer chromatography. Place ethanol abstract of bitter green grass root on silicon G block which contains 1% CMC. Using left-revolving Shikonin as contrast matter to spread out with Toluene-Acetic Ether-Formic Acid (5:1:0.1). Violet spots appear. It turns to blue spots after 10% potassium hydroxide is sprayed on.

The anti tumor effect of shikonin is studied, which shows a positive result. A 5˜10 mg/kg Shikonin (shikonin) extracted from Macrotoria euchroma, is able to inhibit the growth of S₁₈₀ cells of ascites sarcoma. The inhibition rate is above 90%. 10 mg/kg can extend the life-span of the cancerous mouse to 92.5%. Its derivative (shikonin) has activity effect on Walker's cancerous tumor W₂₅₆ and sarcoma S₁₈₀.

The effect of increased sensitivity towards radiation by shikonin is also examined. In connection with Shikonin's observation of magnified sensitiveness of radiation upon the mouse's cancer H₂₂ and Lewis lung cancer, the result reveals, with regards to the cancerous inhibition rate of liver cancer H₂₂ and Lewis lung cancer, the combination of Shikonin and Radiation Group is higher than pure Shikonin or pure radiation (p<0.05). The postpone length of Tumor's growth of the combination of Shikonin and Radiation Group is longer than pure Shikonin or pure radiation. Life-span of the mouse is also longer than the other groups. Under the microscopic observation, a relatively larger necrosis mess is found in the cancerous tissues of the combination of Lewis lung cancer, Shikonin and Radiation Group. Ultrastructure shows the existence of some macrophage is found among the cancerous cells of the combination of Shikonin Group and Shikonin Group plus Radiation Group. The combination of Shikonin and Radiation Group is obvious. It explains Shikonin has a certain magnified radioactive sensitiveness on lung cancer and Lewis lung cancer. Combination of Shikonin and Radiation Group may increase the immediate killing power of Lewis lung cancer's macrophage of the mouse upon the cancerous cells. The extended survival time of the cancerous mouse indirectly strengthens the curative effect.

The anti tumor mechanism of shikonin is analyzed. Study shows, Macrotoria euchro-ma's effective composition (β,β-dimethyl acryloyl Shikonin, β,β-dimethyl propionyl Shikonin, acetyl shikonin, and β-acetoxylation formed acylate Shikonin) has definite effect on the compound's later stage (G2 phase) of DNA of Hela cells, under the condition of culture with 0.25 mg/ml density. Thus, entering the fission is delayed and causes the dropping of fission index. 4 hours after the beginning, the fission index drops by 70%. However, no inhibition effect on the cells absorbing the nucleopyrimidine of 3H-thymus. Therefore, it has no effect on the DNA's composition, whereas it has the effect on basic element like the meadow-saffron in the midterm of fission, but the effect is relatively mild. Thus, Shikonin, not only affects the G2 phase, it is also able to interfere with other segments. Further experiment proves this portion of abstract has more obvious inhibition effect upon EAC, U14, S180, S39 and W 256.

The pharmaceutical kinetics of shikonin is examined. After intravenous injection with H-Shikonin within the mouse's internal metabolism of ³H-mark's Shikonin, half-phase declination period of blood plasma elimination is 8.99 hours. Volume of distribution (vd) is 8.911/kg. After the intravenous injection, content in the bile and liver is the highest, lungs, spleen, kidney, heart and skin the medium, testicle, muscle and brain the lowest. Absorption is rapid after the muscular injection and stomach irrigation. Peak reaching time of blood plasma density is 7.62 minutes. After the intravenous injection, only a small portion discharges with urine and stool in prototype, and most of it discharges in changed form.

The adverse side effect of shikonin is also studied. Shikonin's poisonous side effect is relatively minor. Abstract of Macrotoria euchroma (Royle pauls) ethanol (contains about 56% of Shikonin) has no outstanding effect on mechanism of blood coagulation. Stomach irrigation of the mouse shows no toxicity. Intravenous injection of LD₅₀ is 1.30±0.03 mg/kg. Mouse receives intravenous injection of Shikonin weighs over LD₅₀ mg/10 g. Intravenous injection LD₅₀ of Shikonin mouse is 1430±0.032 g/kg. Stomach irrigation with 2 g/kg shows no dead mouse.

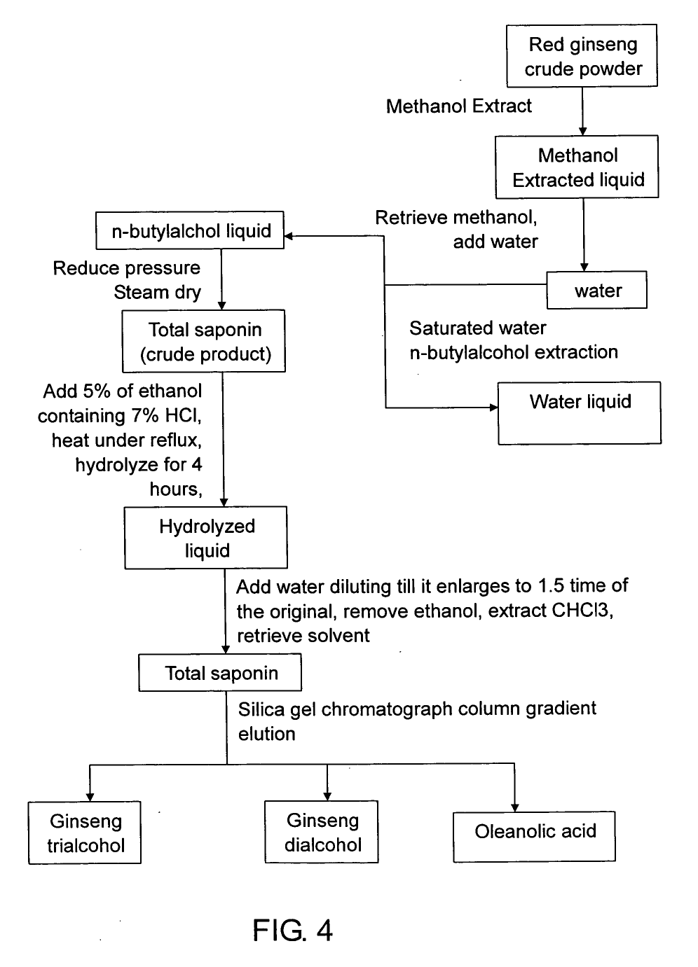

Referring to FIG. 6 of the drawings, an extracting and separating process of shikonin of the present invention is illustrated. Dry root of Arnebia euchroa (Royle) Pauls, Lithospermumerythrohizon Sieb et. Zucc or Arnebia guttata Bunge is immersed in 95% ethanol to form a ethanol fusion. Retrieving the ethanol to form a concentrated liquid, a one third volume of 2% sodium hydroxide is added such that the color is changed from violet to blue and filter the solution into a sediment and a filtrated liquid. The filtrated liquid is then reacted with concentrated hydrochloric acid and further filtered into a filtered liquid and a sediment. The sediment is then washed with water to neutral and dry below 60° C. to form the crude of shikonin. The pure shikonin is then formed by crystallization using benzene repeatedly.

Referring to FIG. 1D of the drawings, the chemical structure of panaxans are shown. After Panaxans A˜U 21 kinds of polysaccharides have undergone repeated separation and purification until various electrophoresis and chromatography are proved to be homogeneity polysaccharide, its structure is, then, analyzed and conclusive. The structure of the three dextrans in the ginseng polysaccharide A are shown in FIG. 1D.

The molecular weight of panaxans A is 14000 and the specific rotation is +187° (0.23). Three kinds of micromolecular polysaccharides PG-I, II and III. PG-I, separated from the ginseng leaf, are white powder, having optical rotation [α]_{D}+120.7° (0.2/H₂O) and molecular weight 2066. Proportionally, they are formed of rhamnose:arabinose:galactose:glucose (0.15:0.7:1:30.87). PG-II is white powder [α]_{D}+9.8° (0.5/H₂O) with molecular weight 2324. It is proportionally formed of rhamnose:arabinose:mannose:galactose:glucose in the ratio of 1:5.07:0.21:20.46:13.03. PG-III is a light yellow powder with [α]_{D}+39.6° (0.5/H₂O). Its molecular weight is 1433 and is formed of rhamnose:arabinose:mannose:galactose:glucose in the ratio of 0.43:1.62:1:4.64:4.36. All three polysaccharides have relatively strong immune function.

Ginseng polysaccharides can improve the immune effect on tumor. The immune effect of ginseng on tumor is the chief effective mechanism of ginseng's fighting tumor. Experiment confirms ginseng polysaccharide is able to enhance the phagocytic function of the reticulate endothelial system. It stimulates the complement and antibody in the blood to grow and increase the content of immunity globulin in the blood. It also inhibits the growth of experimental tumor such as S₁₈₀, EAC U₁₄ and etc. Thus, anti-tumor effect of cyclophosphamide is strengthened. Clinical experiment effect is outstanding.

Experiment shows ginseng polysaccharide is not only restricted to enhancing the anti-tumor function of cyclophosphamide, it is also able to resist the immune toxicity caused by cyclophosphamide. Experiment proves ginseng polysaccharide is able to strengthen the phagocytic function of the reticulated endothelial system of normal animal. It contains stimulating effect in the growth of serum complement, IgG and antibody. Ginseng polysaccharide also has the stimulant effect on the cancerous mouse's phagocytic function of the reticulated endothelial system. Ginseng polysaccharide has no immediate killing effect on cancerous cells. It has no obvious effect on the composition of DNA and RNA of the tumor cells.

Experiment proves ginseng polysaccharide can clearly inhibit the proliferation of cells of Ehrlich's ascites cancer and extend the survival time of the mouse. It has the protection effect on the cancerous mouse and the mouse with weaker immune competence. It has no direct elimination effect on EAC cells. Thus, it is related to the inhibition function of mouse S₁₈₀ sarcoma and EAC cells and the strengthening of organic immunization. Ginseng polysaccharide is able to cause the peculiar plaque forming cells, rosette forming cells, serum anti-SRBC antibody and the antibody secretion of spleen cells, in the cancerous S₁₈₀ mouse, increase significantly. It indicates ginseng polysaccharide is able to enhance the immune ability in the organs of the cancerous mouse.

Referring to FIG. 1E of the drawing, the chemical structure of astragalan is illustrated. Astragalan mongholicus Bunge is the root of leguminous plant. Two types of dextran, AG-1 and AG-2, and two types of heteropolysaccharide, AH-1 and AH-2, are extracted from the astragalus liquid.

The white powder AG-1 dissolves easily in water [α]¹⁵_{D}+171.7° (0.4/H₂O). Its characteristic viscosity in the water is 18.0. Elemental analysis shows it does not contain nitrogen. The infrared spectrum is 1050, 930, 840 and 760 cm⁻¹. Its structure is confirmed to be α-(1→4)(1→6) dextran, α-(1→4) and α-(1→6) glycosidic bond's composition ratio is 5:2.

The water soluble astragalus dextran is obtained from the alkaline extract of the root of astragalus monogholicus Bunge. The molecular weight is 5×10. [α]¹⁶_{D}=+192° (1/H₂O). The elemental analysis shows neither nitrogen nor ash is contained. Thus, it is inferred that the astragalus dextran is the leading chain of α-(1→4) glucose and every 10 repetitive units has one 6-0 phase branched dextran.

Heteropolysaccharide AH-1 is extracted from the aqueous solution of Astragalus mongholicus Bunge. AH-1 is a white powder dissoluble in water. [α]¹⁵_{D}=+126.4° (0.1/H₂O). Its characteristic viscosity in water is 6.2. It contains no nitrogen and the iodine has no reaction. The infrared spectrum is 890, 950, 1020, 1090, 1350, 1410 and 1605 cm. Absorption peak exists. The monosaccharide formation of AH-1 is: aldonic acid, glucose, rhamnose and arabinose. Its mole ratio is 1:0.04:0.02:0.01. AH-2 is a white powder easily dissolves in water. [α]³⁵_{D}=+87° (0.2/H₂O). Its characteristic viscosity in 0.9% NaCl solution is 2.1 and the infrared spectrum is absorbed at 845 cm. AH-1 is able to enhance the phagocytic function of the abdominal macrophage.

Astragalus reflexistipuls polysaccharide has anti-tumor function. For example, it can magnify the effectiveness upon activating cancerous ascites lymphatic cells (CTL). Magnified effectiveness of astragalus reflexistipuls polysaccharide upon activating cancerous ascites lymphatic cells (CTL). About 1500˜2000 ml of ascites are drawn under sterilized condition in the experiment. CTL cells are cultured through centrifuge. Observe the expansion value of CTL cells in the cancerous ascites with reference to 10 cases of taking APS and in 10 cases of not taking APS. The result shows: taking APS can cause CTL cells in the cancerous ascites be easily activated by IL-2. Post-activation time is 24˜48 hours. Before taking it, the activation time is 48˜96 hours. By using APS, the expansion multiple of cancerous ascites CTL cells is 8˜12.8, and by not taking it, the expansion multiple of cancerous ascites cells is 5˜8.5 times. Experiment result reveals: APS can be used to heighten the organic immune function and enhance the organic anti-cancer function.

APS intensifies the effectiveness of human periphery blood's single nucleus cells producing tumor necrosis factors. Study of APS's intensifying human periphery blood's mononucleus cells producing tumor necrosis factors shows further separation of APS is extracted conventionally and through liquid state of colored chart. Its molecular weight is 20000˜25000. It has obvious enhancement effect upon the tumor necrosis factors (TNF) in the external secretion of a normal person and cancerous patient's periphery blood's mononucleus cells (PBMC). It is further discovered, after the PBMC is separated into adherent cells and non-adherent cells, APS has enhancement effect upon TNF a and TNF P.

Adjustment effect of APS upon the internal LAK cells' anti-tumor activity is also examined. APS adjustment effect upon the internal LAK cell's anti-tumor activity. APS mouse of weight 5˜10 mg/kg and abdominal injection C₅₇BL. It is found the polysaccharide is able to outstanding enhance the proliferation of the mouse's spleen cells. Through 125˜1000 U/ml, the spleen cells are induced externally with 2×10⁶/ml for 4 days. Use [¹²⁵I]UdR release to analyze and test the LAK activity. It is found that activity of mouse's spleen cells LAK injected with APS is higher than the Group injected with physiology saline by 70˜120%. External use of IL-2 can lower by 75%. Activity of LAK raises by 120˜200% and external use of IL-2 drops more than 75%.

Besides, Cells' shortage of oxygen is the basic reason which causes the cancerous symptom to take place. When the adaptability of oxygen-shortage cells to environment short of oxygen and the capability to stabilize the cells' environment are damaged, cells will react explosively. Its vicious proliferation will be uncontrollable and develop into cancerous tumor. Furthermore, height of cancer cells' vicious extent is directly proportion to the extent of cells' oxygen shortage.

Study discovers APS has the outstanding endurance capability on a few kinds of oxygen shortage models. It is especially outstanding on the toxicity of potassium cyanide and the oxygen shortage due to ligation of the neck's general artery. It indicates APS is able to reduce the lose of oxygen of the entire body and increase the tissues' ability to endure oxygen shortage. This may be one of the anti-cancerous mechanism the APS has.

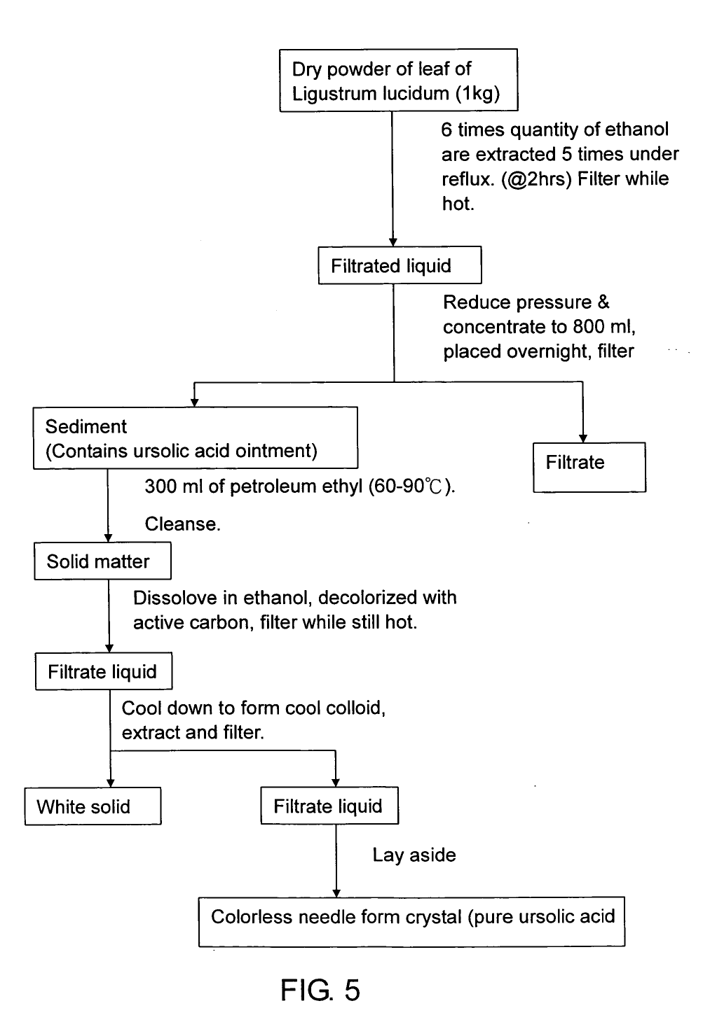

Referring to FIG. 7 of the drawings, an extracting process of astragalan of the present invention is illustrated. APS crude powder is used which undergoes reflux reaction with ethanol and is filtered to form a filtrated liquid and a decoction dreg. The decoction dreg is then filtered after water extraction to obtain a filtrate liquid. The filtrate liquid is concentrated, settled after adding 5 times quantity of ethanol and mixing, and filtered into a sediment and an APS crude powder. The sediment is then completely precipitated and filtered after adding ethanol into another filtrated liquid and another sediment which is then dried to the total polysaccharides (APS).

Referring to FIG. 1F of the drawings, the chemical structure of lentinan (len) is shown. Lentinan is extracted from the hymenium of Lentinus edodes. It is a purified anti-tumor polysaccharide being white to light gray powder. It is stinkless or slightly odorous and tasteless. Lentinan is almost indissoluble in water, methanol, ethanol or acetone, but dissoluble in solution of 0.5 mol/L amino sodium oxide. It contains hygroscopicity. Its molecular weight is about 500,000. Its basic structure is two macromolecular dextran of β-(1→6) combined lateral chain on every 5 β-(1→3) combined gluco-straight chain. Its chemical structure is shown in FIG. 1F of the drawings.

Lentinan is able to shrink various experimental tumors and extend significantly the survival time of tumorous animals. In human body, Lentinan is able to enhance the composition of DNA and the production of peripheral mononuclear leukocyte immunoglobulin.

Anti-Tumor function of the effective portion of Lentinus Edodis (Berk.) Sing is studied. Lentinan polysaccharide and LC-11 separated from Lentinus edodis (Berk.) Sing has higher inhibition rate upon the tumor. The molecule capacity of lentinan polysaccharide is 1 million. [α]²⁰_{D}+19.5°˜+21.5° (1% 2.5N NaOH). It is a kind of 3 polymerized glucose which contains 1β→6 branched chains and 1β→3 branched chains. LC-11 is 1β→4, 1β→6 polymerized glucose. [α]²⁶_{D}−16.3° (1%/H₂O). The anti-tumor function of Lentinan polysaccharide dose not act directly on grafting cancerous cells. It occurs through the adjustment of the host. The curative effect of Lentinan polysaccharide upon the sarcoma 180, CCM cancerous gland (CCM-adenocarcinoma) is very strong. Even a trace can almost recede the tumor completely. By using 5 mg/kg dosage, the inhibition rate of anti-tumor is 97.5%, whereas, with the same dosage of LC-11, the inhibition rate is 93.6%.

The pharmacology of lentinan is also examined. There is effect of lentinan polysaccharide upon the immunity function. Lentinan P. is a polysaccharide of lentinan drawn liquid, the lentinan polysaccharide (KS-2) which is obtained through the process of ethyl sediment. It is mainly the α-manna polymerized sugar. It contains small quantity of serine, threonine, alanine and little peptide formed by amino acid.

Lentinan P. has the function of enhancing the organic immune mechanism. After the female mouse is abdominal-drugged consecutively for 7 days, lentinan p. has outstanding inhibition effect on the mouse's weight and thymus. Heavier dose can reduce the thymus weight but lower dose has no obvious effect. It can increase weight of the mouse's spleen and reduce the amount and density of T cells of thymus cortex area. It has the reinforcement effect on the nucleocells in the multi-nucleus area and the reticulated cells of the splenic sinus. It is able to strengthen the function of cells formed out of antibody and significant enhancement effect on the transformation of external ConA inducing the lymphatic cells of the cells of spleen. It outstandingly increases the phagocytic function of the mouse's reticulated endothelial system and heightens the percentage of circulatory T-lymphatic cells. Its hypha polysaccharide and the subsidiary substance are able to promote the transformation of T-lymphatic cells. Thus, function of the organic immunity of the cells is raised.

Lentinan P. enhances and adjusts the immune mechanism. It has the anti-tumor effect. Its anti-tumor effect lies in heightening the organic immune mechanism. Its main function is to activate the T-cells, therefore, the internal drugging has anti-tumor effect, whereas, the external drugging has no anti-tumor effect from the cultured cancerous cells. Externally, it has no activative reaction on the cultured MΦ and NK cells but internally it has obvious effect. Lentinan P. is able to activate the macrophage and extend the macrophage activity time of the anti-tumor. Conduct intra-abdominal injection of the mouse with lentinan p. 8 mg/kg/daily for 16 consecutive days, and, with the use of mode ₅₁Cr release rate, examine the damage effect of the macrophage on cancerous cells. With ¹²⁵I-Tdr permeate mode, examine the inhibition function of the macrophage on the proliferation of cancerous cells. Using the biochemical method to determine the activity of acetic phosphatase and the arginase in the macrophage. The result shows both the damage effect and the inhibition effect of proliferation of the macrophage are obviously higher than the Contrast Group, and the activity of both enzymes in the macrophage is also higher than the Contrast Group. It manifests lentinan p. can activate the macrophage and intensify its anti-tumor function.

However, promoting the immune activity may not be the only mechanism of lentinan p. to resist tumor. Its best dose to inhibit the S₁₈₀ sarcoma is 1 mg/kg/daily. In fact, it has no anti-tumor effect when the dose is lower than the previously mentioned dosage. When the dosage is higher than the former, inhibiting tumor effect decreases progressively along with the increase of dosage. Moreover, inhibiting tumor function of lentinan p. is closely related to the level of organic endocrine function.

Lentinan p. has a definite anti-tumor effect on the mouse's liver cancer of substantial type and L2 reticulated tissues. Its anti-tumor activity and dosage is not a relationship of direct proportion. Using dosage of 2.5 mg/kg and 5 mg/kg is relatively better, but no obvious curative effect on S₁₈₀.

Upon the secretion of white cell medium-2 (IL-2) and IL-2 receptor (IL-2R), Len has the adjustment function of dicodirection. Len alone has no expression function in stimulating the IL-2 secretion of lymphatic cells and IL-2R. Low density Len works in coordination with PMA and element A23187 has outstanding expression function (p<0.01) in promoting IL-2 secretion and IL-2R. Along with the increase of density, the promotion function also weakens gradually. High density Len finds inhibition function (p<0.01). Len is able to clearly strengthen the capacity of abdominal cavity's macrophage (PMP) secretion IL-1 (p<0.01) of CAPD patient, thereby, the defensive mechanism of the abdomen cavity is improved. It has the function of guarding against the infectious peritonitis.

The toxicity of Lentinan is very mild and has the function of resisting the induced change. Len has very strong inhibition effect on forceful induced change matter, such as methyl formate (A), that stops the DNA reproduction directly. The mouse's orally taken acute toxicity LD₅₀>1250 mg/kg. Drugging mode of 12.5 restraint medication/kg is used in each stomach irrigation of the mouse. After two stomach irrigations, by drugging mode of 17.5 restraint medication/kg, are carried out separately, no harm or death is found. Its long-term toxicity is considerably small. For long-term use, it is considerably safe within the range of clinical treatment capacity. After 50 and 100 times of Len's human injection liquid dose is used respectively, once a day for 6 months consecutively, on dog and large mouse by muscular injection, the appearance, weight, kidney function, blood routine, pathology examination, electronic microscopic observation and etc. are found without extraordinary findings.

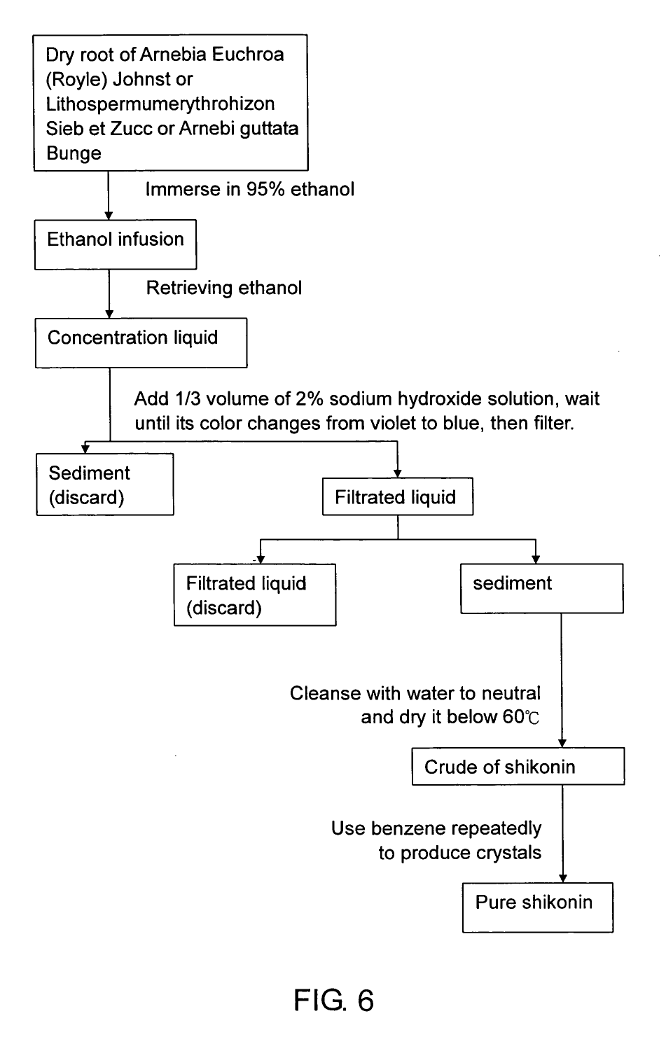

Referring to FIG. 8 of the drawings, an extracting and separating lentinan polysaccharide of the present invention is illustrated. Lentinan is immersed in 5 times volume of hot water for 8 to 10 hours to obtain an extracted matter from hot water, which is then separated into a partial L portion and a partial E portion. The partial E is then separated by 4× ethanol into the sediment EC-11 and the solution EC-14. The partial L is then reacted with CTA-OH (hexadecyltrimethylamine hydroxide) to form a sediment, which is then separated by using 20% HAC into an insoluble portion a soluble portion. The insoluble portion is then dissolved in 6% sodium hydroxide and extracted by 4× ethanol to obtain the sediment Lentinan. The soluble portion is dissolved in 5× ethanol and the sediment is then centrifuged with water such that an upper clean liquid is obtained and separated into LC-1A, LC-1B, LC-1C, LC-1D, LC-1E, LC-11, LC-12, LC-13 by liquid chromatography.

The main physics and chemistry of polysaccharide is further examined. The white powder AG-1 dissolves easily in water [α]¹⁵_{D}+171.7° (0.4/H₂O). Its characteristic viscosity in the water is 18.0. Elemental analysis shows it does not contain nitrogen. The infrared spectrum is 1050, 930, 840 and 760 cm⁻¹. Its structure is confirmed to be α-(1→4)(1→6) dextran, α-(1→4) and α-(1→6) glycosidic bond's composition ratio is 5:2.

Polysaccharide increases along with the polymerization of monosaccharide. Its character is relatively different from the monosaccharide. Normally, it is non-crystalloid without sweet taste, indissoluble in cold water, or becomes colloid solution after it has dissolved in hot water. It is not dissoluble in greasily-related organic solvent. The solubility reduces along with the increase of alcohol density. The cellulose and the chitin are almost indissoluble in any solvent. It has no reductivity. It hydrolyzes and produces oligosaccharide or monosaccharide. They mostly have optical rotation character.

The main chemical reactions are also studied.

Molish Reaction: Dense sulphuric acid and α-naphtholum are the Molish reagent. Reaction of color display principle is the derivative of furfuraldehyde and α-naphtholum condensed to produce colored matter. Reaction out of furfuraldehyde refers to the polysaccharide hydrolyzed in mineral acid to produce monosaccharide and then dehydrated to generate the same product. Furfuraldehyde generated out of pentose is the product. Methyl pentose generates 5-methyl furfuraldehyde and hexatose produces 5-methyldroxyl furufraldehyde. Aldonic acid often turns into furufraldehyde under the decarboxylic condition.

Hydrolysis Reaction:

Acetolysis Reaction: Through the acetolysis reaction, polysaccharide is able to generate acetolyzed monosaccharide and acetolyzed oligosaccharide. Molecules of this lowly polymerized oligosaccharide glycoside bond nature display the composite of the highly polymerized sugar or polysaccharide. This reaction has certain importance in understanding the composition of polysaccharide. Due to the fact that the composition of polysaccharide is, first of all, from the condensed disaccharide or trisaccharide generated out of monosaccharide, then reciprocally condenses to generate highly polymerized matter. In the highly polymerized matter made up of molecules of highly condensed disaccharide or trisaccharide, besides the position of the reciprocal condensation of the monsaccharide and another molecular monosaccharide, the structure of glycoside bond is illustrated. Moreover, the acetic ester of disaccharide or trisaccharide generated out of acetolysis usually has perfect crystal form. Its being apt to separate, extract, refine, examine and confirm is also a merit of this reaction.

Manipulating the acetolysis is relatively simple and easy. Ordinarily, polysaccharide (including oligosaccharide) or acetolyzed polysaccharide dissolved in acetic anhydride or in mixed liquid of acetic anhydride and icy acetic acid is added with little concentrate sulphuric acid (about 3-5%) and placed in room-temperature for 1 to 10 days. After that, the reaction liquid is poured into the icy water, and sodium hydrogen carbonate is added to reach neutralization pH 3-4. With the use of chloroform, acetic ester generated out of monosaccharide and lowly polymerized sugar, is extracted. A unitary composition is obtained after it has been further processed through absorption of the column layer.

Oxidation reaction of periodin acid and its salt: Oxidation reaction of periodine acid is a reaction of oxidize precipitation separation of selective-ness. It can be used in the polysaccharide's molecule group of 1,2 dodehydroxyl and 1, 2, 3 thirteen hydroxyl. that is, the binary alcohol or the triethanol's basic oxidation law of periodine acid in the organic chemistry is applicable in the basic oxidation of polysaccharide's periodine acid.

Thus, after the oxidation of polysaccharide, determine quantitatively the consumption of periodine acid and the proportion between the product of methyl acid and the remainder of sugar. After all, the bond pattern and its proportion of various monosaccharide in the polysaccharide can be determined. Polysaccharide's basic oxidation of periodine acid is usually conducted in the aqueous solution of pH3-5 in the dark. pH too low causes acetic hydrolysis and pH too high leads to non-selective oxidation. Product of dialdehyde type is unstable in the aqueous solution. Therefore, acetic hydrolysis is conducted after the sodium boric tetroxide reduction. Generally, the method, often used, refers to the oxidation of polysaccharide conducted in cold condition with thin periodine sodium. The product of methyl acid and the consumption of periodine sodium are examined at different intervals. Titration or spectrophotometry can be used to determine. Methyl acid is directly determined with the use of standard base burette or indirectly determined by potassium iodide. After the oxidation reaction is totally completed, reduction for more than 10 hours is conducted with the use sodium boric tetroxide, then neutralize and hydrolyze in 100 ¢J thin acid. The hydrolysis product may include a few chemical composites, such as glycerine, ethanol hydroxyl-acetaldehyde, glyceraldehyde, erythrose alcohol, radical monosaccharide and etc. They can be distinguished with paper chromatography, but the distinguishing is more often conducted, after the derivative of trimethyl silicon ethers is manufactured, in the chloric phase layer. Various bond patterns are inferred therefrom.

Smith degradation: Smith degradation is the extending of oxidation reaction of basic periodine acid. It uses the thin inorganic acid to process the controlled hydrolysis (i.e. partial hydrolysis) upon the polyhydrolic alcohol, below room temperature. As a result, various erythrose alcohol glycoside or glycerol glycoside is obtained. Study the composite of these monoglycoside, diglycoside and oligosaccharide offers very important help in expounding the composition of polysaccharide. Sequence of partial joint and bond pattern of monosaccharide in polysaccharide are comprehended therefrom.

Alkali degradation: usually takes place in the ester joining hydroxyl and carboxyl of the monosaccharide. The alkali degradation, which means monosaccharide of the polysaccharide reduction endpoint is peeled off one by one, is often called “the peel off reaction”. It is different from the enzyme degradation reaction. The enzyme degradation reaction occurs at the non-reductive endpoint of the molecules. With the saccharide acid obtained from the analysis of polysaccharide degradation, bond pattern of the original monosaccharide can be inferred.

Enzyme degradation is a selective hydrolysis reaction. Enzyme of different nature can act on different nature's glycoside bond, such as the structural type and the carbohydrate which forms the glycoside bond. Whether or not enzyme of different nature can cause the hydrolysis of monosaccharide or polysaccharide, examine and recognize the resultant of enzyme degradation, indicate the character of glycoside bond in the oligosaccharide or polysaccharide's molecules, it is an important reaction in the study of structural formula of monosaccharide and polysaccharide

Acetic hydrolysis: Usually, the condition to process the complete acetic hydrolysis or controlled hydrolysis is hydrolyzing step by step. In experiment, condition for complete hydrolysis is: 0.5˜3 mol/L H₂SO₄, 100° C. aqueous solution hydrolyzed for 2.5˜8 hours in the sealed tube. Condition for hydrolysis step by step is: 0.1 mol/L H₂SO₄, 100° C. aqueous solution hydrolyzed for 15, 30, 45, 60, 120, 150, 180, or 210 min. Use BaCO₃ or Ba(OH)₂ to neutralize the hydrolysis solution. After the filtrated solution is condensed, it is analyzed with paper chromatography, or ascending mode (single direction or double direction), or descending mode. Select appropriate solvent to develop. Liquid saturated phenol or α-butyl-icy acetic acid-water (BAW) is the frequently used developer.

The extracting and separating polysaccharides is shown. Method of extracting polysaccharide in Chinese herbal medicine is used. Normally, to some extent, it distinguishes along with the different complexity formed by carbohydrate. Due to the fact that polysaccharide is a chemical combination of molecule with maximum nature. It is mostly extracted with the use of different temperature water or thin alkali solution. It would be the best to avoid conducting the extract under the acetic condition, because the acetic condition can cause the breaking apart of the carbohydrate glycoside bond in the polysaccharide. Pay attention to the alkali density and extraction time if alkali is used to extract. Moreover, before the polysaccharide is extracted, sometimes, acetone, ethyl, or ethanol is also used for pretreatment process. It is beneficial for polysaccharide purification. Normally, polysaccharide is extracted from the raw material of post-process by using the boiling water as a solvent. Some of the abovementioned polysaccharide extract liquid can be directly filtrated; some cannot be easily filtrated due to the relatively more sticky extract liquid and often the insoluble have to be removed by way of centrifugal method. Filtrated liquid or upper clear liquid concentrates, added with 2˜5 times ethanol and sediment of crude polysaccharide is obtained. Sediment is cleansed, one after another, with ethanol, acetone, and ethyl. Dry it and powder form crude polysaccharide is obtained. Above is the conventional method of extracting polysaccharide. With the use of this conventional method, crude polysaccharide obtained, often mixes with foreign matters, such as protein, pigment and etc. In order to eliminate the protein, Sevag mode is employed to remove it from the obtained polysaccharide extracted liquid. Sevag mode means using the proportion of n-butyl and chloroform (4:1) to process a few times. In order to eliminate the foreign matter, like the pigment, crude polysaccharide solution passes through the activated carbon column. Some color-borne polysaccharide can also be processed with oxidized decolorization in which concentrated ammonia water is adjusted to about pH8.0 below 50° C. H₂O₂ is added until light yellow color appears. Keep warm for 2 hours. The process is finished.

Experiments are carried out for analyzing the compositions of the present invention. According to a preferred embodiment of the composition of the present invention, the composition A for improving immune system comprises a predetermined quantity of ginsenoside 20(R)-Rh₂, a predetermined quantity of ursolic acid, a predetermined quantity of lentinan, a predetermined quantity of panaxans, a predetermined quantity of astragalan, and a predetermined quantity of lentinan. The molecular formula, molecular weight and effective range are summarized in Table 1.

TABLE 1
Summarized information of the composition according to a preferred
embodiment of the composition A of the present invention.

| | | | | Effective Range |
|---|---|---|---|---|
| | | | Molecular | (Daily dosage |
| No. | Substance | Molecular formula | weight | in mg/kg) |
| 1 | Ginsenoside | C₃₆H₆₂.H₂O | | 0.001-0.1 |
| | 20(R)-Rh₂ | | | |
| 2 | Ursolic Acid | C₃₀H₄₈O₃ | 456.68 | 0.5-10 |
| 3 | Shikonin | C₁₆H₁₆O₅ | 288.31 | 0.5-10 |
| 4 | Panaxans | (CnH2n−2On−1)x | A: 14,000 | 0.01-1 |
| 5 | Astragalan | (CnH2n−2On−1)x | 50,000 | 0.01-1 |
| 6 | Lentinan | (CnH2n−2On−1)x | 500,000 | 0.01-1 |

The preferred embodiment of the composition of the present invention (Composition A) is adapted to be manufactured in different administrative forms, such as tablet, capsule, syrup, solution, powder and sachet form.

The preferred embodiment of the composition of the present invention (Composition A) is used to carry out a variety of experiments for testing. The experimental results are further provided in the follow description.

Experiment 1: The Acute and Chronic Toxicology of Composition A

I. Testing of acute toxicity: 48 large white mouse and 48 small white mouse are selected. The composition A are administered into the stomach of the large white mouse and the small white mouse respectively. The LD₅₀ of the small white mouse is 10.9 g/kg. The GBE dose of the large white mouse is between 12.5 g/kg and 19.5 g/kg. The large and small white mouse are observed for seven days and there is no observable changes in general physical characteristic, appearance, behavior, feeding habit, urine, feces, and body weight.

II. Testing of chronic toxicity: The Composition A, of three dosages namely low, medium, high dosage group (of GBE: 3.5 g/kg, 7 g/kg, and 14 g/kg respectively), is administered into the stomach of large white mouse consecutively for 90 days. During the testing period, the general physical characteristic, appetite, appearance, behavior, activity, etc. of the large white mouse are observed. Body weight is measured every 2 to 4 weeks. Routine blood test, blood platelet and hemoglobin are examined before and after the test. After 90 days, the day after the last administration of Composition A, except a number of large white mouse are used for control and observation, the rest are used for liver and kidney function examination with blood extracted by removing eyeballs, and then are killed to obtain the heart, liver, spleen, kidney, adrenal gland, lungs, small intestine, reproduction organs, etc. for macroscopic observation and histological testing.

The results of the testing of chronic toxicity shows that there is no abnormal changes in appetite, activity, and vigorousness of the low, median, and high dosage groups. The body weight of the medium and low dosage groups are increased significantly. The results also show that composition A exerts no adverse effect on the blood, hemoglobin, liver and kidney function of the large white mouse. The three dosage groups of large white mouse also have no abnormality in pathological examination.

Experiment 2: The Chronic Toxicology of Composition A on Dog

Four groups, namely control, low dose, medium dose and high dose groups, each having four domestic dogs, are divided. The low, medium and high dose groups are administered 20 g/kg, 35 g/kg, and 75 g/kg composition A into the stomachs respectively for a consecutive 180 days.

During the testing period, the appetite, behavior, hairs, etc. of the dogs do not have abnormal changes. Two (2) of the high dose group exhibit lower appetite, decreased activity after administration of composition A for three days, but the appetite and activity are resumed to normal gradually after 10 days. The overall appetite in the low dose group is relatively better and the increase in body weight is more obvious (p<0.05).

Different tests are carried out to observe the effect of the composition A towards the testing groups. The blood tests, including white blood cells, red blood cells, and blood platelets, of the three groups shows that there is no significant changes and that the composition A has no inhibitory effect towards the blood of the dogs. The urine routine test of the three groups shows that there is no signification changes in the physical properties (such as tubular morphology and uric salt). The liver function test shows that there is no significant effect on the liver function amongst the three testing groups. The kidney function test shows that there is no significant effect on the kidney function amongst the three testing groups. The heart rate, electrocardiograph and breathing tests show that there is not significant effect amongst the three testing groups. The histological examination of the heart, liver, lung, kidney, bladder, adrenal gland, cerebrum, intestine, and reproductive gland shows that there is no significant changes.

Selected data of the above in experiment 2 is summarized in Table 2.1, 2.2, 2.3, 2.4, and 2.5.

TABLE 2.1
Effect of the composition A on peripheral blood (X ± SD, ×10³/mm³)

| | Control Group | Low Dose | Medium Dose | High Dose |
|---|---|---|---|---|
| Before testing | 10.21 ± 3.76 | 9.82 ± 2.60 | 10.41 ± 3.26 | 9.81 ± 3.19 |
| 60 days after testing | 9.21 ± 3.55 | 10.20 ± 3.12 | 10.20 ± 1.86 | 9.66 ± 3.21 |
| 120 days after testing | 10.16 ± 3.49 | 9.68 ± 2.99 | 11.69 ± 1.20 | 9.82 ± 2.69 |
| 180 days after testing | 9.98 ± 3.20 | 9.68 ± 2.99 | 11.69 ± 1.20 | 9.82 ± 2.69 |

TABLE 2.2
Effect of the composition A on blood platelet (X ± SD, ×10³/mm³)

| | Control Group | Low Dose | Medium Dose | High Dose |
|---|---|---|---|---|
| Before testing | 212.2 ± 11.26 | 221.1 ± 12.20 | 205.6 ± 10.01 | 208.2 ± 12.11 |
| 60 days after testing | 205.3 ± 10.20 | 212.3 ± 13.20 | 232.8 ± 15.1 | 231.4 ± 10.12 |
| 120 days after testing | 221.2 ± 11.37 | 208.3 ± 13.67 | 250.2 ± 10.28 | 229.6 ± 12.41 |
| 180 days after testing | 208 ± 11.22 | 216.4 ± 11.26 | 246.7 ± 11.45 | 210.5 ± 11.67 |

TABLE 2.3
Effect of the composition A on hemoglobin (X ± SD, g/100 m)

| | | | |
|---|---|---|---|
| Control Group | Low Dose | Medium Dose | High Dose |

| | | | | |
|---|---|---|---|---|
| Before testing | 14.46 ± 1.48 | 14.02 ± 1.56 | 15.20 ± 2.10 | 14.49 ± 1.46 |
| 60 days after testing | 13.92 ± 1.44 | 15.21 ± 1.27 | 14.23 ± 1.79 | 15.20 ± 2.06 |
| 120 days after testing | 14.96 ± 1.76 | 16.23 ± 1.69 | 15.36 ± 2.34 | 14.40 ± 1.69 |
| 180 days after testing | 15.21 ± 1.20 | 16.96 ± 1.16 | 16.37 ± 3.10 | 14.21 ± 1.69 |

TABLE 2.4
Effect of the composition A on liver function (Aspartate AST (u)) (X ± SD)

| | Control Group | Low Dose | Medium Dose | High Dose |
|---|---|---|---|---|
| Before testing | 52.16 ± 42.10 | 50.21 ± 30.28 | 42.37 ± 26.76 | 50.01 ± 26.30 |
| 60 days after testing | 51.20 ± 26.18 | 46.18 ± 20.28 | 40.12 ± 20.26 | 39.36 ± 18.40 |
| 120 days after testing | 48.20 ± 18.21 | 42.16 ± 21.20 | 32.28 ± 17.60 | 40.27 ± 16.96 |
| 180 days after testing | 46.18 ± 30.12 | 40.60 ± 22.26 | 40.02 ± 18.68 | 38.26 ± 20.24 |

TABLE 2.5
Effect of the composition A on urine chlorine (mg %) (X ± SD)

| | Control Group | Low Dose | Medium Dose | High Dose |
|---|---|---|---|---|
| Before testing | 8.9 ± 2.8 | 9.8 ± 4.1 | 9.5 ± 4.4 | 10.1 ± 3.2 |
| 60 days after | 10.0 ± 1.9 | 9.6 ± 5.2 | 8.6 ± 3.4 | 10.6 ± 3.7 |
| testing | | | | |
| 120 days after | 8.3 ± 4.1 | 9.9 ± 3.7 | 9.3 ± 2.9 | 10.7 ± 4.1 |
| testing | | | | |
| 180 days after | 8.6 ± 2.6 | 9.6 ± 4.0 | 9.4 ± 2.6 | 9.8 ± 2.1 |
| testing | | | | |

Experiment 3: Pharmacological Studies on Composition A

3.1: Effect of Composition A on Immune System

3.1A Hemolysis plaque test: Four groups of small healthy mouse are selected, namely control group A, test group A, control group B and test group B. 0.2 ml physiological saline is administered to each subject of the control group A per day for 7 days, 0.2 ml 7% composition A is administered to each subject of the test group A per day for 7 days, 0.1 ml physiological saline is administered to each subject of the control group B per day for 7 days, and 0.1 ml 7% composition A is administered to each subject of the test group B per day for 7 days. The results are shown in Table 3.1A.

TABLE 3.1A
Results of hemolysis plaque test

| | Number of | Mean ± Standard Deviation | |
|---|---|---|---|
| Group | subject | (Hemolysis plaque) | P value |
| Test Group A | 6 | 56.60 ± 5.80 | <0.01 |
| Control group A | 6 | 134.0 ± 9.70 | |
| Test Group B | 6 | 65.60 ± 5.30 | <0.01 |
| Control group B | 6 | 125.0 ± 8.70 | |

The results show that daily administration of 0.2 ml 7% composition A has obvious inhibitory effect upon cell formation of antibody while that of 0.1 ml 7% composition A has no significant effect.

3.1B The test on transformation of lymphatic cell: Four groups of small healthy mouse are selected, namely control group A, test group A, control group B and test group B. 0.2 ml physiological saline is administered to each subject of the control group A per day for 7 days, 0.2 ml 7% composition A is administered to each subject of the test group A per day for 7 days, 0.1 ml physiological saline is administered to each subject of the control group B per day for 7 days, and 0.1 ml 7% composition A is administered to each subject of the test group B per day for 7 days. The results are shown in Table 3.1B.

TABLE 3.1B
Results of test on transformation of lymphatic cell

| | Number of | Mean ± Standard Deviation | |
|---|---|---|---|
| Group | subject | (Hemolysis plaque) | P value |
| Test Group A | 35 | 4210.90 ± 290.50 | >0.01 |
| Control group A | 15 | 2050.20 ± 240.30 | |
| Test Group B | 25 | 3920.38 ± 220.20 | >0.01 |
| Control group B | 20 | 2115.35 ± 196.38 | |

The results show that daily administration of 0.2 ml or 0.1 ml 7% composition A has obvious stimulating effect upon the transfromation of T-lymphocyte.

3.2 Protective Effect of composition A on liver is examined and described as follows:

3.2A Effect of increasing serum transaminase caused by CCL4: Four groups of small healthy mouse are selected, namely control group, CCL4 group, high dose group and low dose group. 0.2 ml physiological saline is administered to each subject of the control group, 0.2 ml 10% composition A is administered to each subject of the CCL4 group per day, 0.2 ml 7% composition A is administered to each subject of the high dose group per day for 7 days, and 0.1 ml 7% composition A is administered to each subject of the low dose group per day for 7 days. CCL4 is administered to each subject of the high dose and low dose groups respectively after 7 days. The glutamic pyruvic transaminase (SGPT) is measured and the results are shown in Table 3.2A.

TABLE 3.2A
Results showing effect of increasing serum transaminase caused by CCL4.

| Group | SGPT u % | P value |
|---|---|---|
| Control Group | 27.6 ± 20 | |
| CCL4 Group | 510 ± 42.8 | |
| High Dose Group | 46.8 ± 72.6 | P < 0.01 |
| Low Dose Group | 57.9 ± 92.3 | P < 0.01 |

The results show that both the high and low dose groups lower the level of increase in transaminase induced by CCL4.

3.2B Protective effect of composition A on liver: Four groups of small healthy mouse are selected, namely control group, CCL4 group, high dose group and low dose group. 0.2 ml physiological saline is administered to each subject of the control group, 0.2 ml 10% composition A is administered to each subject of the CCL4 group per day, 0.2 ml 7% composition A is administered to each subject of the high dose group per day for 7 days, and 0.1 ml 7% composition A is administered to each subject of the low dose group per day for 7 days. CCL4 is administered to each subject of the high dose and low dose groups respectively after 7 days. The subjects of the CCL4 groups are killed 16 hours after administration of the composition A for biopsy study. The subjects of the high and low dose groups are killed after administration of the CCL4 for biopsy study.

The biopsy study shows that the high and low dose group exhibit reduced damage level and thus the composition A has protective effect to liver damage.

3.2C Effect on the increase in serum lipid peroxide (MDA): Four groups of small healthy mouse are selected, namely control group, CCL4 group, high dose group and low dose group. The results are shown in Table 3.2C.

TABLE 3.2C
Results of Composition A on the increase in serum lipid peroxide.

| Group | Dosage of Composition A | MDA (F.U.) | p value |
|---|---|---|---|
| Control Group | | 0.283 ± 0.207 | |
| CCL4 Group | | 0.379 ± 0.190 | |
| High Dose Group | 700 mg/kg | 0.267 ± 0.070 | <0.01 |
| Low Dose Group | 250 mg/kg | 0.273 ± 0.29 | <0.01 |

The result indicates that the composition A is capable of inhibiting the formation of free radical induced by CCL4. Thus, composition A, which comprises a plurality of predetermined compositions carefully selected and combined, is extracted from natural plants and comprises a variety of active substances.

It has the effectiveness to remove the unhealthy and consolidate the foundation; mediate the liver and eliminate the congestion; clear up pyretic and benefit the moist; facilitate with oxygen and replenish energy channels; cure illness and the stasis. It specifically treats cancerous cells of the midterm and the later period. More than 20 years have been spent to obtain the composition A of the present invention, starting from the very basic to the clinical research. Observations of clinically curative effect have been carried out successively during the course of post-cancer surgery of the patient, hepatocellular carcinoma of both midterm and later period, adenocarcinoma pf pancreas, tumor of digestive canal, lung cancer, prostate cancer and etc. Preliminary result indicates the product has a certain curative effect upon the abovementioned tumors. It is especially effective, in a short period of time, to achieve controlling the generally ascending A.F.P. number among the patents of hepatocellular cancer.

Experiment 4: Result of Composition A on Clinical Research

1. Curative Effect of the Survival Period:

This study chooses 270 patients of the II and III stage hepatocellular cancer, 167 male and 103 female. They are randomly selected and divided into 3 groups, namely Chemotherapy Control Group, Composition A Treatment Group and Combined Treatment Group, with 90 people in each group. The period of time for treatment and observation is two years. The survival period is summarized in Table 4.1 below and the survival period analysis is summarized and shown in Table 4.2.

TABLE 4.1

| | | Time Statistics of Survival Period | | | | |
|---|---|---|---|---|---|---|
| | Number | 3 | 6 | 12 | 18 | 24 months |
| Group | (n) | months | months | months | months | or above |
| Chemo- | 90 | 19 | 44 | 18 | 7 | 2 |
| therapy | | | | | | |
| Control | | | | | | |
| Composition | 90 | 3 | 10 | 18 | 33 | 26 |
| A Treatment | | | | | | |
| Combined | 90 | 9 | 23 | 28 | 19 | 11 |
| Treatment | | | | | | |

TABLE 4.2

| | | Curative Effect Statistics of Survival Period | | | |
|---|---|---|---|---|---|
| | | | | | Effective Rate |
| Group | Number (n) | Very Effective | Effective | Non-Effective | (%) |
| Chemotherapy | 90 | 2 | 25 | 63 | 30 |
| Control | | | | | |
| Composition A | 90 | 26 | 51 | 13 | 85.6 |
| Treatment | | | | | |
| Combined | 90 | 11 | 47 | 32 | 64.4 |
| Treatment | | | | | |

The results show that: (i) The curative effect of Composition A Treatment Group is most outstanding. Its effective rate is 85.6%. Effective rate of Combined Treatment Group is 64.4% and Chemotherapy Contrast Group 30%; (ii) Comparison between Composition A Treatment Group and Chemotherapy Control Group has obvious difference (p<0.001); (iii) Comparison between Combined Treatment Group and Chemotherapy Control Group has obvious difference (p<0.01); and (iv) Comparison between Composition A Treatment Group and Combined Treatment Group has obvious difference (p<0.01).

2. Comparison of Cancer Mess and Composition A Treatment

This study chooses 270 cases of the II and III stage patients of hepatocellular cancer. Based on the diameter product's mean of the two largest reciprocal vertical cancer mess, change of size is observed before and after the treatment. Reduction of size of cancer mess can be seen in Composition A Treatment Group, whereas, those seen in other Control Groups enlarge to a certain degree. Comparison between Composition A Treatment Group and Chemotherapy Control Group has relative difference (P<0.01).

The statistics of size change of cancer mess is summarized in Table 4.3 and the curative effect analysis of cancer mess is summarized in Table 4.4.

TABLE 4.3
Statistics of size change of cancer mess

| Group | Number | Pre-Treatment | Post-Treatment |
|---|---|---|---|
| Chemotherapy Control | 90 | 73.16 ± 61.24 | 97.37 ± 77.21 |
| Composition A Treatment | 90 | 80.31 ± 59.11 | 47.26 ± 53.17 |
| Combined Treatment | 90 | 77.37 ± 60.22 | 83.16 ± 56.23 |

According to the WHO standard, the total remission rate of Composition A Treatment Group is 28.9%, compare to Chemotherapy Control Group, there is obvious difference (p<0.01).

TABLE 4.4
Curative Effect Analysis of Cancer Mess

| | | | | | | CR + PR(%) |
|---|---|---|---|---|---|---|
| | | | | | | Total |
| | | CR Complete | PR Partial | SD | SD | Remission |
| Group | Number (n) | Remission | Remission | Stable | Deteriorate | Rate |
| Chemotherapy | 90 | | 2 | 25 | 63 | 2.0 |
| Control | | | | | | |
| Composition A | 90 | 9 | 17 | 51 | 13 | 28.9 |
| Treatment | | | | | | |
| Combined | 90 | 1 | 10 | 47 | 32 | 12.2 |
| Treatment | | | | | | |

3. Lab Test and Other Tests

3.1. Peripheral Hemogram: This is the study of 270 cases of hepatocellular cancer patients. The peripheral hemogram before and after treatment are shown in Table 4.5, 4.6 and 4.7.

TABLE 4.5
Analysis of White Cells Change

| | | ˜4.0 | ˜3.0 | ˜2.0 | ˜1.0 | <1 |
|---|---|---|---|---|---|---|
| | Number | Pre-T | Pre-T | Pre-T | Pre-T | Pre-T |
| | (n) | Post-T | Post-T | Post-T | Post-T | Post-t |
| Chemotherapy | 90 | 70 | 47 | 31 | 27 | 5 |
| Control | | | | | | |
| Composition | 90 | 84 | 89 | 6 | 1 | |
| A Treatment | | | | | | |
| Combined | 90 | 80 | 74 | 9 | 8 | 1 |
| Treatment | | | | | | |

Note:
Pre-T: Pre-Treatment
Post-T: Post-Treatment

TABLE 4.6
Analysis of Red Cells Change

| | | −11.0 | | −9.5 | | −8.0 | | −6.5 | | <6.5 |
|---|---|---|---|---|---|---|---|---|---|---|
| | | Pre-T | | Pre-T | | Pre-T | | Pre-T | | Pre-T |
| | Number (n) | Post-T | | Post-T | | Post-T | | Post-T | | Post-t |
| Chemo- | 90 | 44 | 35 | 28 | 33 | 16 | 21 | 1 | 1 | |
| therapy | | | | | | | | | | |
| Control | | | | | | | | | | |
| BRM- | 90 | 50 | 60 | 29 | 27 | 11 | 3 | | | |
| Synergy | | | | | | | | | | |
| Treatment | | | | | | | | | | |
| Group | | | | | | | | | | |
| Combined | 90 | 44 | 40 | 31 | 29 | 15 | 21 | | | |
| Treatment | | | | | | | | | | |
| Group | | | | | | | | | | |

Note:
Pre-T: Pre-Treatment
Post-T: Post-Treatment

TABLE 4.7
Analysis of Blood Platelet Change

| | | ˜100 | | ˜75 | | ˜50 | | ˜25 | | <25 | |
|---|---|---|---|---|---|---|---|---|---|---|---|
| | Number (n) | Pre-T | Post-T | Pre-T | Post-T | Pre-T | Post-T | Pre-T | Post-T | Pre-T | Post-T |
| Chemotherapy | 90 | 68 | 60 | 18 | 23 | 4 | 7 | | | | |
| Control | | | | | | | | | | | |
| BRM-Synergy | 90 | 70 | 77 | 18 | 13 | 2 | | | | | |
| Treatment | | | | | | | | | | | |
| Group | | | | | | | | | | | |
| Combined | 90 | 69 | 73 | 19 | 17 | 2 | | | | | |
| Treatment | | | | | | | | | | | |
| Group | | | | | | | | | | | |

Note:
Pre-T: Pre-Treatment
Post-T: Post-Treatment

The hemogram (white cell, hemoglobin and blood platelet) of Composition A Treatment Group and the Combined Treatment Group, before and after treatment, has no obvious difference (p>0.05). However, after treatment, the three index of Chemotherapy Control drop. Compare with the Composition A Treatment Group, the difference is outstandingly clear (p<0.001).

According to the above-mentioned experiment 4, it confirms that Composition A does not contain toxic side effect from anti-cancer chemotherapy medication that inhibits the marrow.

2. Urine Routine Examination

This is the study of 270 cases of hepatocellular cancer patients. The urine routine of each section shows no apparent changes before and after the treatment (p>0.05).

3. APT: Transformation of APT in this study of 270 cases of hepatocellular cancer patients. The results are summarized in Table 4.8.

TABLE 4.8
Analysis of APT Transformation (ng/ml)

| | | <50 | | 250˜ | | 500˜ | |
|---|---|---|---|---|---|---|---|
| | | | Post- | | Post- | | Post- |
| | Number (n) | Pre-T | T | Pre-T | T | Pre-T | T |
| Chemotherapy | 90 | 4 | 1 | 28 | 10 | 58 | 79 |
| Control | | | | | | | |
| Composition | 90 | 5 | 75 | 30 | 6 | 55 | 9 |
| A Treatment | | | | | | | |
| Combined | 90 | 3 | 44 | 27 | 19 | 60 | 27 |
| Treatment | | | | | | | |

Note:
Pre-T: Pre-Treatment
Post-T: Post-Treatment

Target of the study is the hepatocellular cancer patients of the II and III stage. Condition of all the patients is critical. Chemotherapy Control Group has practically no control over APT, before and after the treatment.

Before and after treatment, compare the Composition A Treatment Group with Chemotherapy Control Group, the difference is significantly obvious (p<0.01). Compare the Composition A Treatment Group with the Combined Treatment Group, the difference is obvious (p<0.01).

Compare the Combined Treatment Group with the Chemotherapy Control group, the difference is obvious (p<0.01).

4. Analysis of Immune Index: This is the study of 270 cases of hepatocellular cancer patients determined through T cells sub-group. The results are summarized in Table 4.9.

TABLE 4.9
Analysis of Transformation of T cells Sub-Group (mean ± SD)

| | | Treatment Group | |
|---|---|---|---|
| Group | Sub-Group | Pre-Treatment | Post-Treatment |
| Chemotherapy | CD₃ | 55.21 ± 7.29 | 53.10 ± 7.66 |
| Control | CD₄ | 35.34 ± 6.12 | 33.21 ± 8.10 |
| | CD₈ | 26.37 ± 5.19 | 26.58 ± 8.00 |
| | CD₄/CD₈ | 1.34 ± 0.30 | 1.25 ± 0.20 |
| Composition A | CD₃ | 54.90 ± 7.40 | 58.80 ± 7.29 |
| Treatment | CD₄ | 35.80 ± 6.00 | 46.20 ± 8.20 |
| | CD₈ | 26.42 ± 5.76 | 26.60 ± 5.82 |
| | CD₄/CD₈ | 2.07 ± 0.30 | 1.47 ± 0.30 |
| Combined Treatment | CD₃ | 54.88 ± 7.38 | 55.86 ± 6.86 |
| | CD₄ | 36.19 ± 6.38 | 40.20 ± 7.60 |
| | CD₈ | 26.62 ± 5.26 | 26.48 ± 5.50 |
| | CD₄/CD₈ | 1.36 ± 0.28 | 1.52 ± 0.28 |

The result shows that: after the Chemotherapy Control Group treatment, CD₃ and CD₄/CD₈ are slightly lower. Compare to pre-treatment, there is no obvious difference (p>0.05); Composition A Treatment Group is able to raise CD₄, CD₃ and CD₄/CD₈. Compare to pre-treatment, the difference is obvious (p<0.01). Compare to Chemotherapy Control Group and the Combined Treatment Group, the difference is obvious (p<0.01). Comparing the Combined Treatment Group with Chemotherapy Control Group, the difference is obvious.

According to the urine routine test of the experiment 4, the result shows the Composition A Treatment not only does not have the toxic side-effect of immune inhibition from chemical medicines, in fact, it is capable of raising the immune function of primary hepatocellular cancerous cells, causing the increase of specific value of CD₃ (Total T cells), CD₄ (TH) and CD₄/CD₈.

Before and after treatment, examination of every CT finds no new liver transfer or bone transfer.

II. Result of Harmful Reaction

(1) Condition of Liver Function

Condition of liver function: Before the treatment, Chemotherapy Treatment Group has all the 69 cases of the liver function within the range of normal value. After the treatment, 51 cases arise. And, before the treatment, BRM-SYNERGY has all the 67 cases of liver function within the range of normal value. After the treatment, 79 cases are within the normal range. Comparison of the two shows very clear difference (P<0.01).

Before and after the treatment, the Composition A Treatment Group and the Combined Treatment Group, including the comparison of the two group, show no significant difference (p>0.05). It illustrates the composition A does not have side-effect of accidental injury, stem from the vein's chemotherapy medication, on the liver function.

Condition of Kidney Function: Before the treatment, Chemotherapy Control Group has all the 73 cases of liver function within the range of normal value. 64 cases remain after the treatment. There is obvious difference before and after the treatment (P<0.05). And, the composition A group has 68 cases of the liver function within the range of normal value. After the treatment, 83 cases arise. Comparison between the Composition A Treatment and Chemotherapy Control Group has obvious difference (P<0.01). There is no obvious changes between the composition A Treatment Group and the Combined Treatment Group before and after the treatment. Comparison of the two groups shows no obvious difference (p>0.05). It proves the two groups having the Composition A treatment do not have side effect of accidental injury, which stems out of the vein's chemotherapy medication, on the liver function.

(2) Electrocardiogram Examination

Before the treatment, Chemotherapy Control Group's electrocardiogram examination has 74 cases within the range of normal value. After the treatment, 61 cases are within the range of the normal value, whereas 78 cases of Composition A Treatment Group are normal. After the treatment, 87 cases are normal. Comparison of the two groups has very obvious difference (p<0.01). Before and after the treatment, comparison between the Composition A Treatment Group and the Combined Treatment Group has no obvious difference. It points out that the composition A has no damaging side effect to the heart. Due to the fact that the vein's chemotherapy treatment has the adriamycin in its treatment program, therefore, compare to the composition A Treatment Group, obvious damaging effect to the heart is clearly shown (p<0.001).

(3) Effect Upon the Peripheral Blood Cells and Immune System

1. No effect on the peripheral blood cells. 2. No inhibition effect on the immune system.

(4) Effect Upon the Digestive System and Bodily Condition

During the drugging period, nausea, vomit, abdominal distention and decline of appetite are not found. Patients do not appear to have bodily unwell and insanity during the treatment period.

(5) Allergic Reaction

During the drugging period, allergic reaction such as allergic shock and skin rash has not surfaced.

Experiment 5: Pharmacology Research of the composition A is studied. The inhibitory effect on tumor and the stimulating effect on immune system is analyzed.

I. Inhibitory Effect on Tumor:

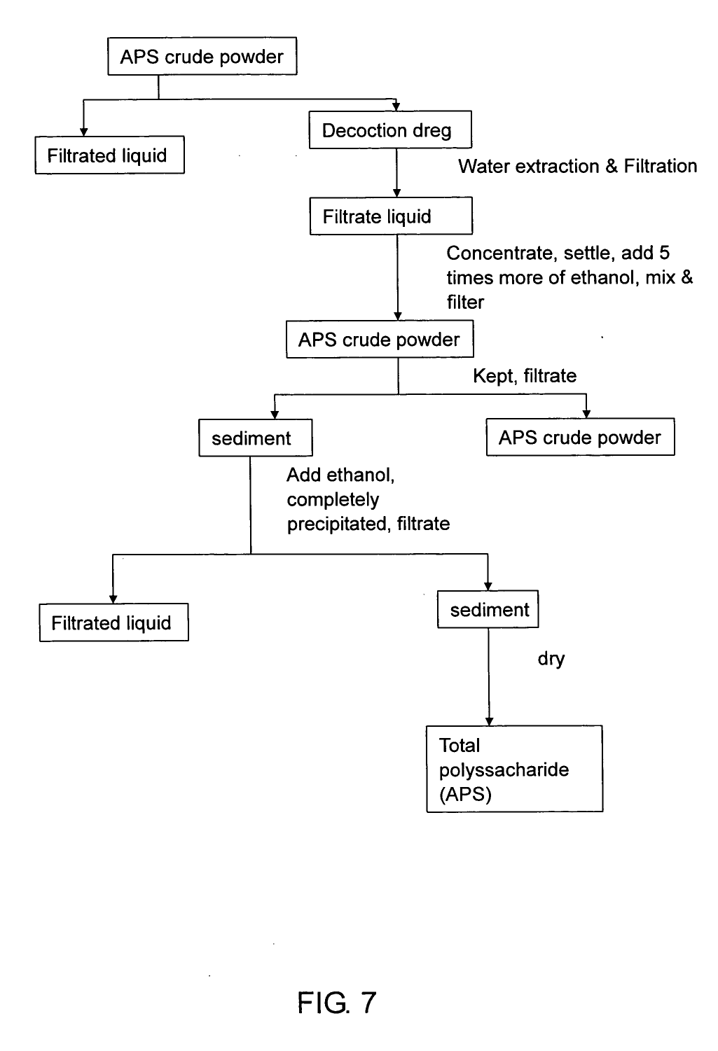

(1) The administration of composition A has inhibitory effect on mouse. The inhibitory effect on tumor S180 of mouse of the administration of composition A is substantially higher than the control group under chemotherapy treatment. The composition A, and the podophylloacetaldehyde and paraplatinium, of medium and small dose (p<0.05) are used for comparison and the effect is further illustrated in FIG. 9 of the drawings.

(2) The inhibitory effect of the composition A and chemotherapy with (podophylloacetaldehyde and paraplatinium), with three dosage levels, on substantial tumor of the mouse S180 is examined. The results are summarized in Table 5.1.

TABLE 5.1

| | | Number of | Average | | |
|---|---|---|---|---|---|
| | | Mouse | Tumor | Rate of | |
| | | (Start/ | Weight (g) | Inhibition | p |
| Group | | End) | X ± SD | (%) | Value |
| 0.9% NS | | 30/29 | 4.06 ± 0.56 | — | |
| High Dose | Composition A | 30/29 | 2.48 ± 0.41 | 39.23 | <0.01 |
| | Chemotherapy | 30/15 | 3.15 ± 0.56 | 22.35 | |
| Medium | Composition A | 30/30 | 1.68 ± 0.32 | 58.60 | <0.01 |
| Dose | Chemotherapy | 30/21 | 3.39 ± 0.41 | 16.20 | |
| Low Dose | Composition A | 30/30 | 2.94 ± 0.49 | 30.02 | <0.01 |
| | Chemotherapy | 30/26 | 3.51 ± 0.41 | 11.01 | |

(3) The effect of the administration of composition A on the growth of tumor volume of cancerous mouse S1180 (X±SD) is studied and the results are summarized in Table 5.2.

TABLE 5.2

| | 1st Group | | 2nd Group | |
|---|---|---|---|---|
| | Number of | | Number of | |
| | mouse | Tumor Weight | mouse | Tumor |
| Group | (Start/End) | (mg) | (Start/End) | Weight (mg) |
| Contrast | 20/8 | 2216.2 ± | 20/7 | 1674.32 ± |
| Group | | 610.30 | | 686.30 |
| Drugged | 20/19 | 721.31 ± | 20/18 | 545.34 ± |
| Group | | 416.15** | | 603.20** |

(**p < 0.001)

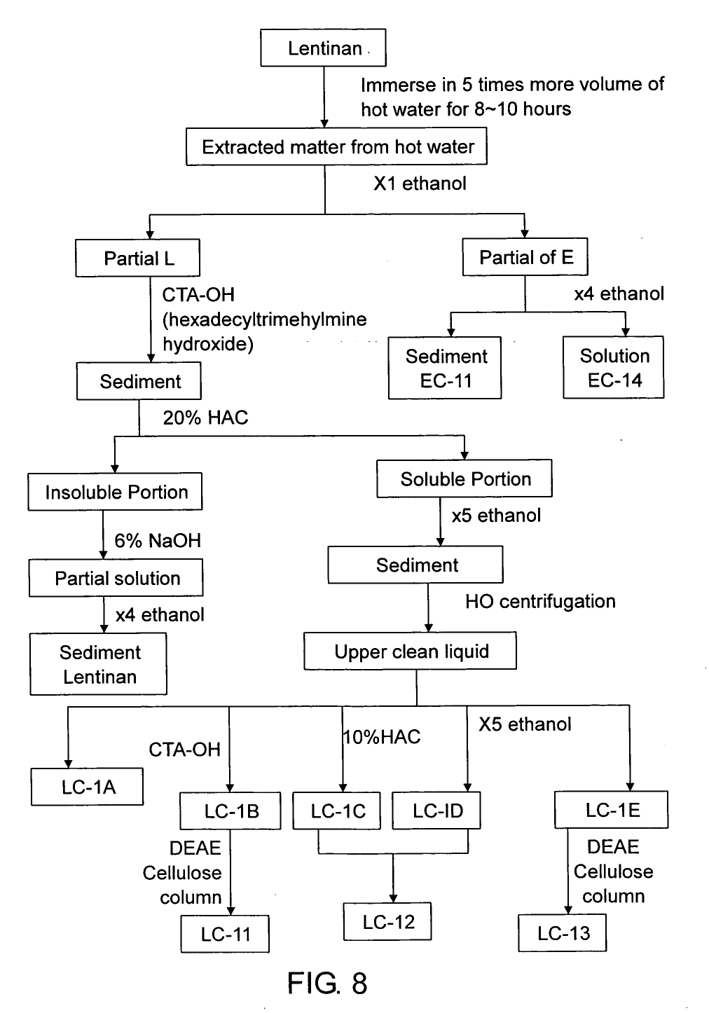

(4) The inhibitory effect of administration of composition A on mouse with Ehrlich's Ascites Cancer is studied, and the results are summarized in FIG. 10 of the drawings. The life extension rate is examined and compared, and it is shown that the average life span of the mouse in high, medium and low dose groups having the administration of composition A is significantly longer than those having chemotherapy treatment of cyclophosphorus amines respectively.

(5) The inhibitory effect of administration of composition A on mouse with Ehrlich's Ascites Cancer is studied, and the results are summarized in Table 5.3.

TABLE 5.3

| | | Number of | Average | | |
|---|---|---|---|---|---|
| | | mouse | Tumor | Rate of | |
| | | (Start/ | Weight (g) | Inhibition | p |
| Group | | End) | X ± SD | (%) | Value |
| 0.9% NS | | 30/27 | 20.20 ± 3.98 | — | |
| High Dose | BRM Synergy | 30/29 | 4.75 | 76.50 | <0.01 |
| | Chemotherapy | 30/25 | 14.68 | 27.35 | |
| Medium | BRM Synergy | 30/30 | 3.57 | 82.35 | <0.01 |
| Dose | Chemotherapy. | 30/27 | 14.10 | 30.26 | |
| Low Dose | BRM Synergy | 30/29 | 8.06 | 60.12 | <0.01 |
| | Chemotherapy | 30/26 | 16.11 | 15.32 | |

II. The stimulatory Effect of the Composition A on Immunity

(1) The effect of Stomach administration of the composition A on cAMP/cGMP of Mouse's cancerous tissue is studied. The treatment group with the administration of the composition A and the control group without treatment with composition A are compared and the results are summarized in Table 5.4.

TABLE 5.4

| | First Group | | Second Group | |
|---|---|---|---|---|
| | Number of | | Number of | |
| | mouse | | mouse | |
| Group | (Start/End) | cAMP/cGMP | (Start/End) | cAMP/cGMP |
| Control | 20/8 | 0.07 ± 0.42 | 20/11 | 0.13 ± 0.16 |
| Treatment | 20/18 | 0.16 ± 0.01 | 20/19 | 0.17 ± 0.03 |

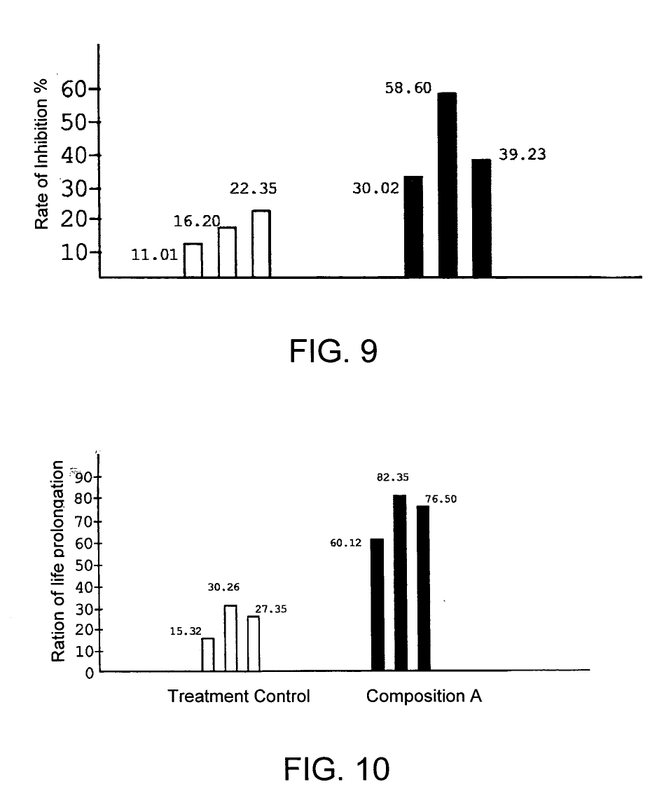

(2) The effect of the composition A on activity of NK cells is studied. The effect of the treatment group with administration of composition A, the interference group with administration of interference substance, and the control group with administration of physiological saline are compared, and is shown in FIG. 11 of the drawings. The results show that the administration of the composition A has stimulatory effect on the activity of natural killer cells.

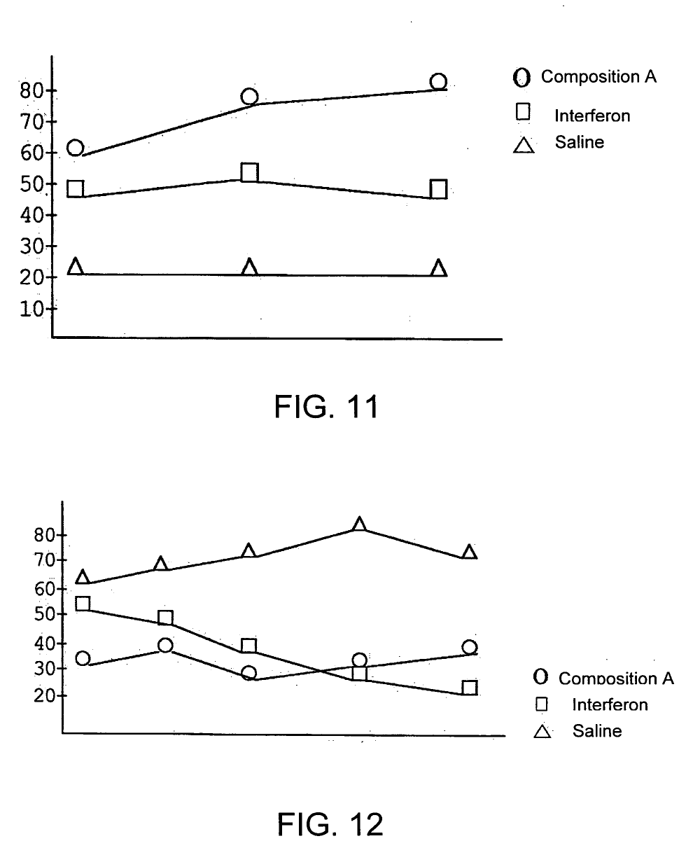

(3) The effect of the composition A and interference substance on phagocytic function of macrophage are studied. A control group with administration of physiological saline is used. The results are as shown in FIG. 12 of the drawings.

Referring to FIG. 12 of the drawings, the composition A has stimulatory effect on phagocytic function of macrophage, that is the composition A is capable of improving the activity of macrophage and is capable of maintaining the immune system in an active state.

Experiment 6: The composition A is also used for testing the effect on active NK cell and IL-2 activity of cancerous spleen of mouse.

One of the most commonly seen malignant tumors that seriously menaces the health and life of mankind is the cancer of liver cells. Low status of the immunity of organism is closely related with the cause and development of liver cancer. It is clinically proved that patients of liver cancer often have low active NK cells and IL-2. Adjustment effect of both upon the T-cell's immunity is significantly important. Thus, for the last few years, active NK cells and IL-2 level have been heavily valued in the study of cause and development of tumor and in the theory of monitoring the immunity adjustment. This study is to monitor the two norms through the symphysis experiment of animal, and to further explore the relation of both and the clinical significance of fighting cancer.

1. Experiment Materials

1.1 Experimental Animal: Mice of 6 to 8 weeks old, with equal number of male and female, each having a weight 20±2 g is obtained by the U.S. Experimental Animals Center for the experiment 6.

1.2 Cell Stem: Cell stem of cancerous liver cell is used.

1.3 Test substance: Composition A, extracted through specific technology (listed separately) and purification, in 600 mg capsule form furnished by Young America Inc.

2. Experiment Method

2.1 Modeling: Subcutaneous inoculation method using suspension fluid of cancerous liver cell. Liver of cancerous mouse is peeled off under sterile condition and is used to prepare suspension fluid of cancerous cells. The suspension fluid is further concentrated into 1×107/ml. Each of the abdomens of the subjects of the experiment is then localized sterilized and each received a subcutaneous injection of 0.2 ml concentrated suspension fluid.

2.2 Grouping and Treatment of Testing Subjects

40 mice are randomly selected and divided into Control Group (having 10 mice) and Cancerous Group (having 30 mice). Each subject of the Control Group receives abdominal subcutaneous injection of 0.2 ml physiology saline. Each subject of the Cancerous Group receives abdominal subcutaneous injection of 0.2 ml concentrated suspension fluid prepared from tumor cells of cancerous liver. Cancerous Group is divided into two groups, namely treatment control group and treatment group, with 15 mice in each group.

The treatment control group is fed with normal food without treatment. The treatment group receives 0.7 ml/10 g composition A by stomach irrigation, at a frequency of three times per day, for a total period of 48 days. The control group is fed with normal food without treatment and the subjects are killed after 48 days for removing eyeball, extracting blood, and obtaining spleen.

2.3 Testing of Active NK Cells in Spleen:

(1) Preparation of NK cells of spleen: Spleen of mouse is obtained at bacterium-free condition for preparing a suspension fluid of spleen cells. Centrifuge the suspension and the sediment containing lymphatic cells is obtained. Complete culture fluid 1640 containing 10% FCS is then put into a 100 ml cell culture flask. Culture under 37° C. and 5% CO₂ for 4 hours and adjust the concentration to 6×10⁶/ml for further use.

(2) Target cell: Change fluid a day before the experiment to perform hand-down generation culturing YAC-1 cells. Density is adjusted to 3×10/ml.

(3) Determine the active killing nature of NK cell: Use culture block with 96 holes, and dividing the specimen into four groups, namely a, b, c, and d. Group a is spontaneity release hole of effect cell (effect cell of spleen 0.1 ml, complete culture fluid 0.1 ml); group b is sponateity release hole of target cell (target cell 0.1 ml; culture fluid 0.1 ml); group c is experiment hole of damage (target cell 0.1 ml, spleen effect cell 0.1 ml); and group d is largest experiment hole of damage similar to c. Establish 3 duplicate holes for every hole; centrifuge; discard 0.1 ml upper clear fluid; add 0.1 ml 2% x-100; culture 24 h under 37° C. and 5% CO₂; centrifuge (1,000 rpm, 5 min); discard upper clear fluid; add 100 μl NAG enzymogenic reactive fluid; having been placed in the 37° C. damp box for 40 min warm breeding, add 100 μl suspension fluid to each hole, measure A value at 410 nm point of zymogen monitor. Indicate with a, b, c, and d accordingly. Compute NK cells according to the following formula:

$$Cytotoxin index ( CI ) = \frac{( a + b ) - c}{( a + b ) - d} \times 100 \%$$

2.4 Testing of IL Activity

(1) Preparation of spleen cell as ditto (2.3 (1) above).

(2) Reviving IL-2: Place 6×10⁶/ml spleen cell suspension fluid into culture block with 24 holes; adjust cell density to 10 μg/ml; culture 48 hours (1,000 rpm, 10 min) under 37° C. and 5% CO₂; collect upper clear fluid; sterile synthetic-tip tube as specimen pending for test; kept in −20° C. for activity testing.

(3) Testing Activity: Set IL-2 dependent cellular stem CTLL-2 as target cell. Use 1640 culture fluid of 20% FCS to adjust 2.5×10⁵/ml density. Place into culture block with 96 holes. Each hole has 100 μl. Again, add 100 ul specimen pending for test. Set 3 duplicate holes for each specimen and also negative contrast hole. After culturing for 48 hours, centrifuge (at 200 rpm, 10 min) the specimen. Discard Upper clear fluid. Add 100 μl NAG enzymogenic reactive sediment. Place in a 37° C. damp box to warm breed for 48 minutes. Add 100 μl suspension fluid to each hole. Measure specimen A value at 410 nm point of zymogen monitor. Computer CTLL-2 proliferation rate according to the following formula to show IL-2 activity.

$$proliferation rate ( \% ) = \frac{Specimen A value - negative contrast hole A value}{Negative contrast hole A value} \times 100 \%$$

2.5 Method of Computation: Use t Test

3. Analysis of Results

3.1 Effect of the composition A upon spleen NK cell of mouse and IL-2, is as shown in Table 6.1.

TABLE 6.1
Effect of composition A upon Spleen NK Cell of Mice and
IL-2 (X ± S %)

| | | Damage Rate | |
|---|---|---|---|
| | (N) | of Spleen | Proliferation Rate (%) of |
| Group | Cases | NK Cell | CTLL-2 |
| Control Group | 10 | 43.20 ± 7.98** | 20.67 ± 4.38* |
| Treatment Control | 15 | 20.26 ± 4.96 | 15.21 ± 3.26 |
| Group | | | |
| Treatment Group | 15 | 41.76 ± 5.98** | 27.69 ± 4.66** |

Notes:
Compare to Treatment Control Group
*P < 0.05
**P < 0.01

3.2 Transformation of Spleen NK Cell Activity

NK cell activity of Treatment Group and IL-2 production are obviously stronger than Treatment Control Group. Its difference is also very obvious (p<0.01). NK cell activity is obviously reduced (p<0.001) from the comparison between Treatment Control Group and Control Group. It also shows the NK activity of cancerous mice and production of IL-2 are significantly lower than normal, whereas, composition A is capable of clearly heightening the activity of both. Experiment shows that the composition A is able to raise the NK activity of spleen cell of mouse and improve the IL-2 production level in the peripheral serum.

4. Discussion

NK cell is a very important immune adjustment cell. It possesses the function of anti-tumor, anti-infection, immunity adjustment and etc. Its mechanism of anti-tumor is: (1) Through its surface receptive, target structure of tumor cells is distinguished and killed directly, or, eliminate tumor cell through the release of dissoluble medium. (2) Surface of NK cell has FcγR receptive that is capable of integrating the Fc section antibody covering the tumor cell and elaborating the cytotoxin effect within the tumor cell. (3) NK cell is able to release cellular factors, such as interference element γ(IFN-γ), Il-1 and IL-2 to enhance the anti-tumor function. NK cell is at the forefront line of defense in the realm of fighting organic tumor. It has important implication in terms of inhibiting the growth, development and metastasis of tumor.

IL-2 has significant effect upon tumor immunization. Its mechanism is: (1) stimulate the growth of NK cell and improve the NK cell's activity of soluble cell; (2) Induce the function of differentiation and effectiveness of tumor killer cells such as CTL, LAK and etc.; (3) induce the production of tumor killer cells such as IFN-γ, TNF-a and etc., and intensify the function of anti-tumor; and (4) Activate the plasma cells and improve the effect of eliminating tumor. Clinically, IL-2 injection is often used to inhibit the tumor. It has definite curative effect.

Surface of the composition A has IL-2 receptive. Reaction of cell proliferation takes place when IL-2 is stimulated and leads to produce cellular factor such as IFN-a. IL-2, IFN-a and IFN-γ are able to work in coordination with one another to heighten the activity of NK cell. The function of IL-2 is most outstanding. Mutual interaction of NK cells and IL-2 elaborate the function of killing tumor.

The pharmacology composition of the composition A, including Panaxans and Astragalan, is capable of strengthening the immunity of cell. It improves the IL-2 production. Its extract is able to promote the activity of NK cell. Experiment of the animal's internal body confirms that Ginsenoside Rg1 and Rh2 in composition A is able to raise the activity of NK cell and the production level of interference element. Also, it strengthens the immune mechanism, intensifies the anti-tumor rate of the mouse and reduces the tumor's weight. Composition of shikonin and Lentinan in composition A can raise the organic white cells, improve competence of the organic immunity, inhibit growth of the tumor and reduce size of the tumor.

Experiment shows composition A is able to strengthen the activity of NK cell and the ability of T cell to secrete IL-2. It raises the revival level of interference element. As a result, the immune mechanism of organism is improved. Patients of tumor, especially patients in the state of progress, whose T cell secretion IL-2 level and the activity of NK cell are lower than normal people. Thus, composition A is able to heighten the patient's immune competence through raising the activity of NK cells and reviving IL-2 level. Consequently, it leads to curative effect upon cancerous liver.

Experiment 7: Study of the Red Cell Immune Effect and Internal Anti-Tumor Mechanism of the Composition A

Summary: This study is about the research of the red cell immune effect and internal anti-tumor mechanism of the composition A. The result indicates composition A is able to clearly improve the immune index of red cells and strengthen the immunity mechanism of organic red cells. Its mechanism of internal anti-tumor and its enhancement of red cell immunity is related to one another. Also, the improved effect of the medication upon the red cell immunity is achieved through the strengthening of red cell's function (not increasing or decreasing of quantity). In fact, it has certain relation with the treatment time.

Experimental study of the composition A, with reference to the effect and mechanism of internal anti-tumor, discovers that composition A is outstandingly effective to inhibit growth speed of the cancer borne by the mouse, prolong the life of the cancerous mouse and enhance the white cell's immunity of the cancerous mouse. Furthermore, in order to explore the effect of composition A upon the immunity of the cancerous mouse and the mechanism of internal anti-tumor, this experiment uses EL-4 cellular stem of the tumor to establish C₅₇BL/6 cancerous mouse model, and observe the adjustment function of composition upon the immunity of the cancerous mouse's red cells.

1. Materials and Method

1.1 Subjects and Grouping: 180 healthy C₅₇BL/6 mice, having no limit to sex, each weight 20±2 g (obtained from Animal Research Institute), under room temperature 20±-2° C. The subjects are randomly selected and divided into Normal Control Group, Treatment Control Group, Normal control treatment Group and Treatment Group. Each group has 45 mice.

1.2 Stem cell: The cancerous stem cell is C₅₇BL/6 thymus cancerous cell stem—EL-4 stem cell of mouse.

1.3 Testing substance: Composition A is extracted with special technology (listed separately) and obtained through purification. The composition A is supplied by YOUNG AMERICA INC. in 600 mg capsule form.

1.4 Establishing C₅₇BL/6 model of cancerous mouse: Inoculate culture the EL-4 cancerous stem cell. When the existence rate of computed cells is lower than 95% under microscopic counting, physiology saline is employed to adjust the cellular density to 1×10 count/ml. Subcutaneous injection of suspension fluid of cancerous cell is performed on the back of the mouse in each group respectively (each receiving 0.2 ml). One injection is carried out in total. The above procedures are manipulated under sterile condition.

1.5 Administration Method: The composition A is prepared into 0.7 g/ml. After filtration and sterilization, the composition A is kept at 4° C. Each mouse of Treatment Group is administered 0.4 ml/10 g composition A by stomach irrigation, at a frequency two times per day for a consecutive 7 days. Normal Control Treatment Group starts administration of composition A five days before the model making process. Treatment Group starts administration of composition A five days after the model making process. Normal Control and Treatment Control Group are administered with equal volume of sterile physiology saline. Subjects of each group are raised conventionally.

1.6 Testing the Immunity Function of Red Cell

After the experiment (the 15th day of the experiment), 15 mice is randomly selected from each group (total 45) for examining the total red cell, red cell ICR and C_{3b}RR and promotion rate of red cell towards the white cell's phagocytic activity, 15 mice is selected randomly for examining the activity of red cells RFER and RFIR, and 15 mice is selected randomly for examining the activity of red cells SOD. Eyeballs of every mouse are excised and blood is drawn. Immunity index of red cells of every item is examined. Sum total of red cells is conventionally computed with cytology counter. The calculation of Floralcyclization rate (RBC·CRI) of the immunity compound of red cells and floralcyclization rate (RBC·C_{3b}RR) of C_{3b} receptive is known and shown in Reference 1. The calculation of promotion rate of red cells to the white cell's phagocytic activity is known and shown in Reference 2. Immunity promotion factor of red cell (RFER) and the activity of inhibition factor (RFIR) using promotion rate of floralcyclization of red cell C_{3b} receptive and inhibition rate to examine the activity of red cell RFER and RFIF respectively (as shown in Reference 3). Activity of ultraoxides of red cell, and dichotomous enzyme (SOD), is known and shown in Reference 2, which employs the inhibition method of triphenol self-oxidation.

2. Result: Test results of red cell 's immunity of mouse in each group is shown in Table 7.1.

TABLE 7.1
Effect of Composition A on the Red Cell's Immunity of Cancerous Mouse
(N = 15 mice) (X ± S)

| | | | Normal | |
|---|---|---|---|---|
| | | | Control | |
| | Normal Control | Treatment | Treatment | Treatment |
| Item | Group | Control Group | Group | Group |
| Red Cell Total | 8.51 ± 1.68 | 8.58 ± 2.03* | 8.41 ± 2.26*Δ | 8.40 ± 2.60*Δ |
| (×10/L) | | | | |
| Red Cell ICR | 12.08 ± 2.04 | 16.77 ± 1.89*** | 12.98 ± 1.39*ΔΔ | 13.08 ± 2.03**ΔΔ▴ |
| (%) | | | | |
| Red Cell C_{3b}RR | 14.65 ± 1.68 | 8.23 ± 2.10*** | 13.07 ± 2.37*ΔΔ | 11.43 ± 2.17**ΔΔ▴▴ |
| (%) | | | | |
| Promotion Rate | 46.10 ± 10.29 | 16.67 ± 9.24*** | 39.66 ± 9.31*ΔΔΔ | 34.02 ± 10.11**ΔΔ▴ |
| (%) | | | | |
| Activity of Red | 1118.5 ± 312.6 | 310.2 ± 211.3*** | 919.8 ± 220.6*ΔΔΔ | 878.6 ± 200.6**ΔΔ▴ |
| Cell SOD | | | | |
| (μ/gHb) | | | | |
| Activity of Red | 50.21 ± 8.20 | 27.23 ± 10.26*** | 47.92 ± 8.36*ΔΔ | 41.37 ± 9.38**ΔΔ▴▴ |
| Cell RFER(%) | | | | |
| Activity of Red | 22.42 ± 11.29 | 39.67 ± 8.11*** | 24.91 ± 9.66*ΔΔ | 30.98 ± 9.90**ΔΔ▴▴ |
| Cell RFIR(%) | | | | |

Notes:
(1) Compare to Normal Control Group *P > 0.05, **P < 0.05, ***P < 0.01;
(2) Compare to Treatment Control Group ΔP > 0.0.5, ΔΔP < 0.05, ΔΔΔP < 0.01;
(3) Compare to Normal Control Treatment Group ▴P > 0.05, ▴▴P < 0.05

Results of Table 7.1 show that comparing to normal control group, treatment group and normal control treatment group have significant differences (with p<0.05 or p<0.01) in all the testing items except the red cell total (p>0.05), all results, except the promotion rate which is obviously lower than the Normal Control Group (p<0.05), of Normal Control Treatment Group, are close to the Normal Control Group (p>0.05). Comparing to the Treatment Control Group, all results of Normal Control Treatment Group, except Red Cell Total (p>0.05), show an outstanding difference (p<0.05 or p<0.01). In Treatment Group, only the Promotion Rate and Activity of Red Cell SOD have outstanding difference (P<0.05 or P<0.01). Comparing to Normal Control Treatment Group, activity of Red Cell C_{3b}RR, RFER and RFIR in the Treatment Group have outstanding difference (P<0.05), whereas, index of the rest has no obvious difference (p>0.05).

3. Discussion

With reference to the mechanism of the composition A against the internal anti-tumor, we have experimentally confirmed that it has significant enhancement effect upon the proliferation capacity of T cells of C57BL/6 mouse and phagocytic effect of plasma cells. It is believed the internal anti-tumor effect of the medication has very much to do with the enhancement of immunity effect of white cells. In order to further study its enhancement effect of immunity and its mechanism of internal anti-tumor, this experiment has observed the impact of the composition A of 500 mg/kg stomach irrigation upon the red cell immunity function of the cancerous mouse. The result shows (compare to Normal Control Group) the immunity function of red cell in the Treatment Control Group is obviously disorderly low (p>0.05 or p<0.01). All indexes in the Normal Control Treatment Group, except the Red Cell Total, have significant improvement (p<0.05 or p<0.01). Most of the index is close to the Normal Control Group (p>0.05). It reveals immunity function of the red cells in the Treatment Control Group is critically damaged. This medication is clearly able to improve the damaged immunity function of the red cells of the experimental animal.

Immunity of the red cells is a very important component of the organic immune system. Composition A's experiment has confirmed red cells have the ability to distinguish, adhere, concentrate and eliminate the internal circulatory immune composite and is able to adjust the organic immunization. Red cell total is the foundation of red cell immunity. Red cell C₅₇BL/6 and ICR is the important index to reflect the adherence of immunity and the function of eliminating foreign matter. Red cell RFER and RFIR is the adjustment factor of the adherence function of serum red cell C_{3b} receptive. The former promotes the adherence function, the latter has the inhibition effect on the red cell adherence function. Red cell SOD is the important oxidizing free medium of elimination enzyme of the organ. It has the immunity function of protecting the tissue cell and strengthening the white cell (including the T cell, K cell, NK cell, LAK cell, plasma cell and etc.) Engulf promotion rate of the red cell reflects the enhancement effect of the red cell upon the immunity function of the white cell. The above-mentioned functions of red cell have very important implication in organic capturing, inflicting casualties on and eliminating cancerous cell. Looking at the result of this experiment, the above mentioned immune index of red cell of the cancerous mouse in Normal Control Treatment Group is improved significantly. It points out the medication has the effect of strengthening the immunization of organic red cell. It is inferred the integration of the medication's internal anti-tumor effect and the immunity of red cell upon the organic tumor resisting are related. Red cell total of each group has no obvious discrepancy. This indicates, through the experiment, the improvement of the immunity effect of red cells is achieved by enhancing the function of red cells (not by increasing or decreasing the volume). Compare the Normal Control Treatment Group with the Treatment Group, index of red cell C_{3b}RR, RFER and RFIR activity has outstanding difference (p<0.05). It points out the medication's immunity effect of red cells has certain relation with the drugging time. Perhaps, the elaboration of organic immunity effect requires a certain course. It brings guiding significance to the medication in terms of clinical application.

Reference for Experiment 7:
1. Edited chiefly by G U O Feng and L O Yongzhen. [Exploring The Immunology of Red Cell. First Edition. Nanjing University Publisher. 1993: pp 155˜157]
2. Compiled and written by LIU Jingtian and ZHANG Jie. [Immunology of Red Cell. First Edition. Shanxi Science & Technology Publisher. 1995: pp 27˜29]

3. G U O Feng. [Determination Method of Red Cell Immunity and Its Adjustment Function. Immunology Journal. 1990, 6 (1):60]

The composition A is attained by accurate and balanced recombination of high efficiency elements that are selected from n extensive array of medicinal herbs through a meticulous process utilizing methods developed by modern cell biology. In the process, harmful biological alkaline is removed form the elements through the balanced recombination; thereby making them harmless to the normal cells and achieving a high level of therapy not obtainable by the ordinary singular herbs.

The composition A is a natural medicine that principally heightens the human immunology. It has been shown by cellular biology that the composition A is a precis combination of many anti-cancer substances, extracted from natural plants, which can induce changes in cancer cells. These substances are high efficiency elements extracted through a repeated process, which has been validated by reverse tumor experiments. Their anti cancer nature is tied to the production of conduction interferons inside the body. They distinctly raise the phagocytic rate and the phagocytic index by stimulating the macrophage and the natural macrophage, increasing the number of the macrophage, enlarging the cells, and raising the phagocytosis. These high efficiency elements share highly compatible with the lipid diatomic m and are capable of rapidly entering the lipid diatomic m. of the cell membrane. By changing the nature of lipid diatomic m. these elements cause the multi function cancer cell membrane molecules to change the messages sent to cell nuclei. The messages in turn lead to a reversal of cancer causing genes such as the c myc. Cancer cells treated by the high efficiency matter gain near normal characteristics of cells; and the division of the cell is suppressed. The cancer cells lose their biological character as the cancer cell nuclei go through degenerative changes such as nucleus breakdown, shrinkage, and vacuolization. The result is an important breakthrough in cell biology research in the world. For example, the augmentation of alpha fetoprotein AFP which is found in most liver cell cancer patients. The series of immunoloigcal anti cancer drugs of young can restore AFP to the normal level in a relatively short period of time within a few weeks. Such drastic reduction proves the radical change sin the nature of the nucleus of the liver cancer cell.

Treating cancer with immuno biology is the most scientific and most effective regimen with the least side effects, it will be the cardinal direction of cancer research for a long time to come. Its superiority is apparent: immuno therapy strengthen the regeneration and resistance capabilities of the reconstructive system of the body; if is the most basic and fundamental therapy. The ingredients of the composition A are active matters obtained from natural herbs. The balances recombination eliminates certain side effects that are common to singular medicines, and, at the same time, massively raises the effectiveness of the combined usage. It also avoids the toxic side effect of large scale physical degradation that accompanies chemotherapy. Coupling BRM and other specific immunological anti cancer drugs the survival and development conditions of cancer cells are essentially eliminated; and the rate of recurrence is lowered. This is particularly effective on primary carcinoma and recurrence carcinoma.

The composition A has the following features in treating cancer: generally, results are brought about in four weeks, each treatment cycle is three months; they may be taken by cancer patients of any primary carcinoma or metastatic carcinoma cancer; they may be used with western medicines, including radiotherapy and chemotherapy; no toxic side effects and non-addictive; and they may be used in the long run for ailment preventing and health maintenance. The composition A is for various immunological disorder syndromes chronic fatigue syndrome, various forms f cancer and to reverse the undesirable effects of immune and blood generation suppression caused by chemotherapy or radiotherapy.

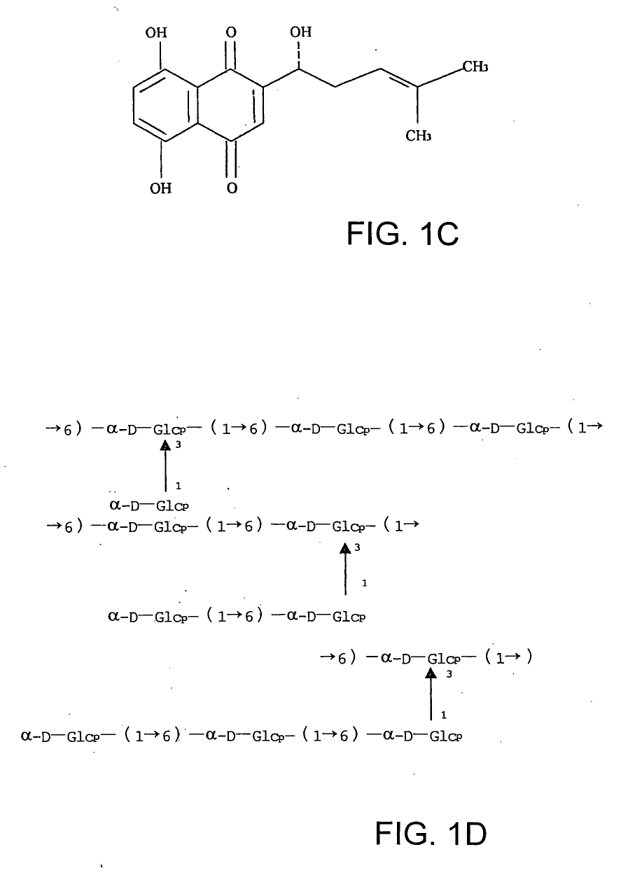

Referring to FIG. 2 of the drawings, a semi-synthetic process of ginsenoside 20(R)-Rh₂ of the present invention is illustrated, wherein the semi-synthetic process comprises the steps of (a) reacting panaxadiol with acetobromo-α-D-glucose; and (b) heating to obtain a resulting compound of ginsenoside 20(R)-Rh₂.

The structure of ginsenoside-Rh₂ is identified as ginseng dialcohol-3-O-β-D-glucopyranoside, which is a white powder, having melting point 264˜266° C., [α]²⁵_{D} positive (0.61/methanol), Liebermann-Burchard reaction positive (+), Molish reaction positive (+), and [IR(KBr)cm⁻¹]: 3400, 2790, 1450, 1385, and 1365.

The acetobromo-α-D-glucose, having the molecular formula C₁₄H₁₉BrO₉ and molecular mass 411.20, has the chemical structure as shown in FIG. 2 of the drawings.

The physical and chemical properties of acetobromo-α-D-glucose are further studied. The acetobromo-α-D-glucose is a white to yellow crystal or powder, which is readily dissolved in water. 1 g of the compound is soluble in 20 ml anhydride ethanol. It is easily soluble in ethyl, chloroform, propanone, ethyl ester and benzene, slightly soluble in petroleum ether. It is generally stabilized by adding 1%-2% calcium carbonate. Its melting point is 88-89° C., optional rotation is: [α]¹⁹_{D}+199.3° (3%/chloroform), and [α]¹⁹_{D} is +230.3° (9%/benzene).

Acetobromo-α-D-glucose is obtained by a reaction of glucose and acetic anhydride, which is then acrylated and bromodize, and crystallized by adding propanone. The resulting crystallized substance is then filtered, washed and dried to obtain the product. It is usually used as the intermediate of glycoside and disaccharide synthesis. The quality standard is its optional rotation [α]²⁰_{D}+190°-196° (3%/chloroform).

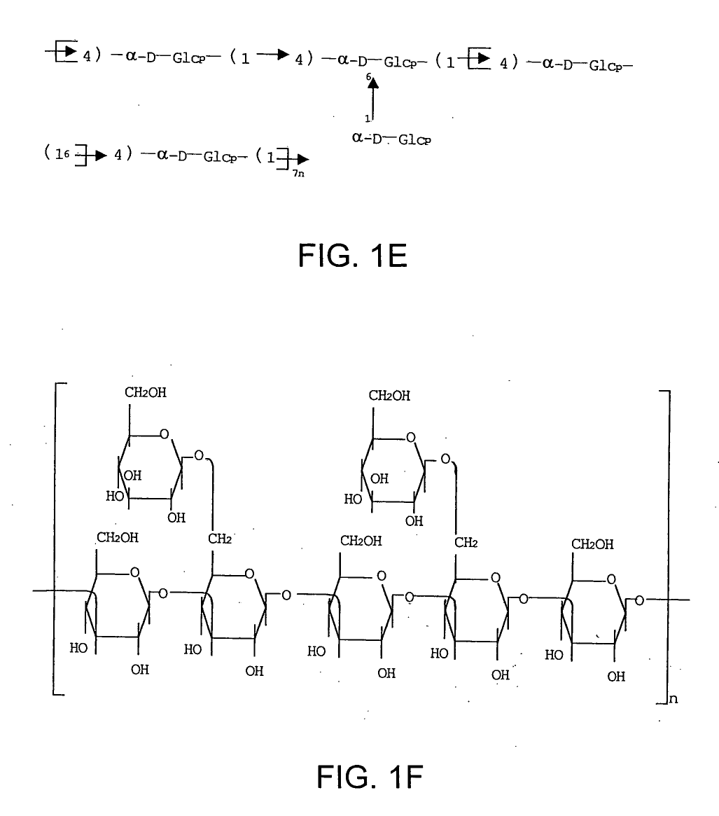

Referring to FIG. 3 of the drawings, a reaction process of 20(S)-protoganaxadiaol (Saponin A) to panaxadiol of the present invention is illustrated. The process comprises the steps of: reacting saponin A with acid to form 20(R)-protopanaxandiol, and heating the resulting protopanaxandiol to obtain the panaxadiol.

There are three types of ginsenoside: the panaxadiol (saponin A); the panaxatriol (saponin B), and the oleanic acid (saponin C).

Among these three types of saponins, the saponin A, having the glucoside 20(S)-protopanaxadiol is the most common type, the saponin B, having the glucoside 20(S)-protopanaxatriol is the next common type, and the saponin C, having the glucoside oleanic acid is the least common type.

Classification and major chemicals of saponins are further examined. The aglycone of both saponin A and saponin B belong to the group of the dammarane having tertracyclic triterpenes which is the derivative of dammarenediol. The structural characteristic is that above the C8 there is an angular methyl substitution which is C13 as β-H; the configuration of C20 is mostly S pattern.

The difference of both lies within the three carboxylic substitutions (C3, C12 and C20) on the parent nucleus-A, the four carboxylic substitutions (C3, C6 and C12) on the parent nucleus-B. The anomer-A is chiefly on C3 and C20. The sugar group is usually glucose (glu), rhamnose (rha) or xylose (xyl). B-pattern is mainly on C6 and C20. Its sugar is mostly glu, rha and xyl. Furthermore, malonic acid monoacetyl base substitution (it can only be obtained from the white ginseng) is found on the sugar of saponin-A and the acetyl base substitution (only to be obtained from the red ginseng). Aglycone of ginsenoside-C is pentacyclic triterpene oleanane which is called oleanolic acid. Its structural characteristic is C28 as monocarboxyl base and combines with sugar to form ester glycosidic linkage. And, glucuronic acid is found in the C3 annectent sugar-chain.

Character of these sapogenin is not very stable. C20 configuration transforms easily to R in the acid hydrolysis and the side-chain cyclized when heated. The cyclization mechanism bases on Markovnikoff's rule. H on C20-OH is added to the side-chain double bond's carbon which contains relatively more hydrogen. C20-O is added to the side-chain double bond's carbon which has lesser hydrogen. Panaxadiol is produced. The course of reaction is as shown in FIG. 3.

Thus it can be seen, when the saponin chemical compound is added with acid and heated to obtain the sapogenin, the panaxadiol is already an isomeric compound. The real oleanane of panaxadiol saponin is 20(S)-protopanaxadiol. There are three carboxyl in the 20(S)-protopanaxadiol molecule, of which the C3-OH, C20-OH and sugar combined to produce glycoside. Due to the fact that C₂₀ carboxyl is tertiary carboxyl, the combination of sugar and acetic acid heated together is hydrolyzed immediately. To hydrolyze the glycoside formed out of C3-OH and sugar, stronger acidic condition is required. C12-OH is free carboxyl. Due to the spatial steric is larger, the cabinet respiration method cannot methylize it.

Physics and chemical characteristics, such as solubility, hemolysis, hydrolytic reaction of the saponins are also examined.

The solubility of ginsenosides generally follows the general solubility of saponins and that the three types of ginsenoside exhibit slightly different solubility. The ginsenoside C having a carboxyl group with greater polarity, and is easily dissolved in water and alkaline water. The polarity of ginsenoside-A is generally lower than the ginsenoside C, but its polarity increases if it contains malonic acid monoacetyl. Like the C type, it is highly soluble in water. The polarity of ginsenoside B is the lowest due to the smaller number of sugar groups in the structure. If secondary glycoside is produced from the hydrolysis of ginsenosides A and B, it is more difficult to dissolve in water but is more readily dissolved in ether.

Total saponins of ginseng generally has no hemolysis characteristic. After a process of separation of the total saponins, the separated ginsenoside B or C has stronger hemolysis tendency. The separated ginsenoside A exhibits antihemolysis function.

Hydrolysis reaction of the saponins is studied. Due to the instability of sapogenin of ginsenoside A and B, the product of acid hydrolysis is panaxadiol and panaxatriol, whereas the real glucoside cannot be obtained.

For many years, panaxadiol and panaxatriol have been thought to be the sapogenin of ginsenoside. Later, after intensive experimental study, with the use of Smith Degradation method, the 20(S)-protopanaxadiol and 20(S)-protopanaxatriol are obtained, which are the genuine sapogenin of ginsenoside.

The hydrolysis characteristic of ginsenoside lies even within the difference of —OH nature of the sugar-chain condensation in its structure, such as the parent nucleus C20-OH is tertiary alcohol group. Its formed glycosidic linkage is easily hydrolyzed. Generally, its hydrolysis takes place when heated together for 4 hours in the 50% acetic acid solution at 70° C. It produces monosaccharide-chain saponin.

Glycosidic linkage formed out of C3-OH, C6-OH and sugar can only be hydrolyzed under the stronger acidic condition. However, the terminal saccharide of saccharide-chain can be hydrolyzed by bond cleavage under the relatively bland condition and the secondary glucoside is produced.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure form such principles. Therefore, this invention includes all modifications encompassed within the spirit and scope of the following claims.

1. A ginsenoside 20(R)-Rh₂ composition for improving the immune system, comprising a predetermined quantity of ginsenoside 20(R)-Rh₂, a predetermined quantity of ursolic acid and a predetermined quantity of shikonin.

2. The composition, as recited in claim 1, wherein said quantity of ginsenoside 20(R)-Rh₂ has an effective range between 0.001 mg/kg and 0.1 mg/kg, said quantity of ursolic acid has an effective range between 0.5 mg/kg and 10 mg/kg, and said quantity of shikonin has an effective range between 0.5 mg/kg and 10 mg/kg.

3. The composition, as recited in claim 1, further comprising a predetermined quantity of panaxans, a predetermined quantity of astragalan, and a predetermined quantity of lentinan.

4. The composition, as recited in claim 2, further comprising a predetermined quantity of panaxans having an effective range between 0.1 mg/kg and 1 mg/kg, a predetermined quantity of astragalan having an effective range between 0.1 mg/kg and 1 mg/kg, and a predetermined quantity of lentinan having an effective range between 0.1 mg/kg and 1 mg/kg.

5. The composition, as recited in claim 1, wherein said ginsenoside 20(R)-Rh₂ is obtained by the steps of:
(a) obtaining a predetermined quantity of ginseng dialcohol from an extraction process of ginseng dialcohol; and
(b) reacting said quantity of ginseng dialcohol with a predetermined quantity of acetobromo-α-D-glucose.

6. The composition, as recited in claim 5, wherein said extraction process of ginseng dialcohol comprises the steps of:
(a) extracting a predetermined quantity of n-butylalcohol from a red ginseng in a crude powder form with methanol;
(b) obtaining a predetermined quantity of total crude saponin from the n-butylalcohol under reduced pressure and steam dry conditions;
(c) refining the total crude saponin by adding 5% ethanol with 7% hydrochloric acid, heating under reflux hydrolysis for 4 hours and cooling under room temperature such that a hydrolyzed solution is obtained;
(d) extracting a total refined saponin from the hydrolyzed solution by diluting the hydrolyzed solution with water to form a diluted solution having 1.5 times volume of the hydrolyzed solution, removing the ethanol, and extracting by CHCl₃; and
(e) separating a predetermined quantity of ginseng trialcohol, a predetermined quantity of ginseng dialcohol, and a predetermined quantity of oleanolic acid from the total refined saponin by gradient cleansing using silica-gel column chromatography CHCl₂—MeOH (8:2).

7. The composition, as recited in claim 3, wherein said ginsenoside 20(R)-Rh₂ is obtained by the steps of:
(a) obtaining a predetermined quantity of ginseng dialcohol from an extraction process of ginseng dialcohol; and
(b) reacting said quantity of ginseng dialcohol with a predetermined quantity of acetobromo-α-D-glucose.

8. The composition, as recited in claim 7, wherein said extraction process of ginseng dialcohol comprises the steps of:
(a) extracting a predetermined quantity of n-butylalcohol from a red ginseng in a crude powder form with methanol;
(b) obtaining a predetermined quantity of total crude saponin from the n-butylalcohol under reduced pressure and steam dry conditions;
(c) refining the total crude saponin by adding 5% ethanol with 7% hydrochloric acid, heating under reflux hydrolysis for 4 hours and cooling under room temperature such that a hydrolyzed solution is obtained;
(d) extracting a total refined saponin from the hydrolyzed solution by diluting the hydrolyzed solution with water to form a diluted solution having 1.5 times volume of the hydrolyzed solution, removing the ethanol, and extracting by CHCl₃; and
(e) separating a predetermined quantity of ginseng trialcohol, a predetermined quantity of ginseng dialcohol, and a predetermined quantity of oleanolic acid from the total refined saponin by gradient cleansing using silica-gel column chromatography CHCl₂—MeOH (8:2).

9. The composition, as recited in claim 1, wherein said ginsenoside Rh₂ is ginseng dialcohol-3-O-β-D-glucopyranoside.

10. The composition, as recited in claim 5, wherein said ginsenoside Rh₂ is ginseng dialcohol-3-O-β-D-glucopyranoside.

11. The composition, as recited in claim 3, wherein said ursolic acid is obtained by an extraction process of ursolic acid comprising the steps of:
(a) washing a predetermined quantity of dry leaf of Ligustrum lucidum with ethanol;
(b) filtering to obtain a sediment containing ursolic acid; and
(c) dissolving the sediment in ethanol and facilitating crystallization to obtain a crystal form of pure ursolic acid.

12. The composition, as recited in claim 3, wherein said shikonin is obtained by an extraction process comprising the steps of:
(a) using alcohol to obtain a concentrated extract from a predetermined specie of plant;
(b) reacting the extract with 2% sodium hydroxide and filtering out a filtered liquid;
(c) reacting the filtered liquid with a concentrated acid to facilitate solidification and filtered out a sediment; and
(d) washing the sediment with water and drying below 60° C. such that a crude shikonin is obtained.

13. The composition, as recited in claim 12, wherein said composition is in a form adapted for administration selected from the group consisting of capsule, tablet, powder, sachet, syrup, and solution.

14. A method of ginsenoside 20(R)-Rh₂ synthesis, comprising a step of:
reacting a predetermined quantity of ginseng dialcohol with a predetermined quantity of acetobromo-α-D-glucose such that a quantity of ginsenoside 20(R)-Rh₂ is obtained.

15. The method, as recited in claim 14, further comprising an extraction process of ginseng dialcohol comprising the steps of:
(a) extracting a predetermined quantity of n-butylalcohol from a red ginseng in a crude powder form with methanol;
(b) obtaining a predetermined quantity of total crude saponin from the n-butylalcohol under reduced pressure and steam dry conditions;
(c) refining the total crude saponin by adding 5% ethanol with 7% hydrochloric acid, heating under reflux hydrolysis for 4 hours and cooling under room temperature such that a hydrolyzed solution is obtained;
(d) extracting a total refined saponin from the hydrolyzed solution by diluting the hydrolyzed solution with water to form a diluted solution having 1.5 times volume of the hydrolyzed solution, removing the ethanol, and extracting by CHCl₃; and
(e) separating a predetermined quantity of ginseng trialcohol, a predetermined quantity of ginseng dialcohol, and a predetermined quantity of oleanolic acid from the total refined saponin by gradient cleansing using silica-gel column chromatography CHCl₂—MeOH (8:2).

16. The method, as recited in claim 14, wherein said ginsenoside Rh₂ is ginseng dialcohol-3-O-β-D-glucopyranoside.

17. The method, as recited in claim 15, wherein said ginsenoside Rh₂ is ginseng dialcohol-3-O-β-D-glucopyranoside.

18. The method, as recited in claim 14, further comprising a step of adding a predetermined quantity of ursolic acid and a predetermined quantity of shikonin into said ginsenoside 20(R)-Rh₂.

19. The method, as recited in claim 15, further comprising a step of adding a predetermined quantity of ursolic acid and a predetermined quantity of shikonin is added into said ginsenoside 20(R)-Rh₂.

20. The method, as recited in claim 18, further comprising a step of adding a predetermined quantity of panaxans, a predetermined quantity of astragalan, and a predetermined quantity of lentinan into said ginsenoside 20(R)-Rh₂.

21. The method, as recited in claim 19, further comprising a step of adding a predetermined quantity of panaxans, a predetermined quantity of astragalan, and a predetermined quantity of lentinan into said ginsenoside 20(R)-Rh₂.

22. The method, as recited in claim 18, wherein said quantity of ginsenoside 20(R)-Rh₂ has an effective range between 0.001 mg/kg and 0.1 mg/kg, said quantity of ursolic acid has an effective range between 0.5 mg/kg and 10 mg/kg, and said quantity of shikonin has an effective range between 0.5 mg/kg and 10 mg/kg.

23. The method, as recited in claim 19, wherein said quantity of ginsenoside 20(R)-Rh₂ has an effective range between 0.001 mg/kg and 0.1 mg/kg, said quantity of ursolic acid has an effective range between 0.5 mg/kg and 10 mg/kg, and said quantity of shikonin has an effective range between 0.5 mg/kg and 10 mg/kg.
